SEQUENCE LISTING (3 sequences)

SEQ ID NO: 1 - protein, 237 aa, Canavalia ensiformis
ADTIVAVELDTYPNTDIGDPSYPHIGIDIKSVRSKKTAKWNMQNGKVGTAHIIYNSVDKRLSAVVSYPNADSATVSYDVDLDNVLPEWVRVGLSASTGLYKETNTILSWSFTSKLKSNSTHETNALHFMFNQFSKDQKDLILQGDATTGTDGNLELTRVSSNGSPQGSSVGRALFYAPVHIWESSAVVASFEATFTFLIKSPDSHPADGIAFFISNIDSSIPSGSTGRLLGLFPDAN

SEQ ID NO: 2 - protein, 385 aa, Gallus gallus
GSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 3 - protein, 124 aa, Bos Taurus
KETAAAKFERQHMDSSTSAASSSNYCNQMMKSRNLTKDRCKPVNTFVHESLADVQAVCSQKNVACKNGQTNCYQSYSTMSITDCRETGSSKYPNCAYKTTQANKHIIVACEGNPYVPVHFDASV

(19) United States
(12) Patent Application Publication
(10) Pub. No.: US 2014/0350370 A1
(43) Pub. Date: Nov. 27, 2014

(54) GLUCOSE SENSING ASSAY

(71) Applicant: The Texas A&M University System, College Station, TX (US)

(72) Inventors: Brian M. Cummins, Raleigh, NC (US); Gerard L. Coté, College Station, TX (US)

(73) Assignee: THE TEXAS A&M UNIVERSITY SYSTEM, College Station, TX (US)

(21) Appl. No.: 14/248,043

(22) Filed: Apr. 8, 2014

Related U.S. Application Data
(60) Provisional application No. 61/809,771, filed on Apr. 8, 2013.

(51) Int. Cl. G01N 33/66; A61B 5/145; G01N 33/58

(52) U.S. Cl. CPC G01N 33/66; G01N 33/582; A61B 5/14532
USPC 600/365; 536/54; 530/395; 435/188; 530/367; 436/501

(57) ABSTRACT

The disclosure provides a ligand that competes with glucose for binding the protein Concanavalin A (ConA) and competitive binding assays incorporating the ligand. The competing ligand binds to the primary and part or all of the extended binding sites of Concanavalin A. These and other aspects of the disclosure are useful for glucose monitoring (e.g., continuous glucose monitoring (CGM)).

### CROSS-REFERENCES TO RELATED APPLICATIONS

This application claims the benefit of Provisional Application No. 61/809,771, filed Apr. 8, 2013, which is expressly incorporated herein by reference in its entirety.

### STATEMENT OF GOVERNMENT LICENSE RIGHTS

This invention was made with Government support under 1RO1DK095101 awarded by the National Institutes of Health (NIH). The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 44022_Sequence_Final_{—}20140407.txt. The text file is 7 KB; was created on Apr. 7, 2014; and is being submitted via EFS-Web with the filing of the specification.

### FIELD OF THE INVENTION

The present invention relates to a glucose sensing assay based on competitive binding.

### BACKGROUND

Diabetes is a disease that is characterized by the body's inability to maintain normal blood glucose concentrations. The body is unable to enact the normal feedback mechanism to convert excess glucose into glycogen, and thus, it is characterized by elevated blood glucose concentrations. As a result, the patient is more likely to form advanced glycated end-products, which can cause complications in tissues/organs with relatively long-lived proteins/cells. There are two primary types of diabetes. Type 1 diabetes is an autoimmune disorder that results in the destruction of the beta cells in the pancreas and is often referred to as juvenile diabetes or insulin-dependent diabetes. This type results in an absolute insulin deficiency where insulin is no longer released at concentrations that can help control the concentrations of glucose. Type 2 diabetes is characterized by insulin resistance due to lifestyle and genetic factors. Lifestyle factors that increase the risk of developing Type 2 diabetes include obesity, lack of physical activity, poor diet, and stress. Of the total number of diabetes cases, approximately 10% have Type 1 and 90% have Type 2.

This chronic disease is currently at epidemic proportions in the United States and around the world. Diabetes affects more than 300 million people worldwide, is at epidemic levels, and there is currently no cure. In 2012, the direct and indirect expenditures associated with diabetes totaled 548 billion U.S. dollars. These numbers are expected to continue to rise. Half of the adult population in the U.S. is expected to have pre-diabetes or diabetes in 2020. By the year 2035, there are estimates that diabetes will affect 592 million people worldwide.

Patients are typically instructed to manually control their blood glucose concentrations to manage treatment and to decrease complications. The blood glucose meter is the primary tool to perform these measurements. Briefly, the device requires a drop of blood to be applied to an enzyme-coated paper strip that is then inserted in a handheld device to be measured. The enzymes on the paper strip consume the glucose, converting it into byproducts that are typically measured electrochemically. The device uses an algorithm to convert this signal into an expected blood glucose concentration, and these readings typically show an error that is less than 20%. Possible sources of increased error include the denaturation of the enzymes on the paper strip, inadequate blood volumes, and variations due to temperature. Physicians typically instruct patients that use insulin to make 5-7 measurements per day (before and after meals) to get a true picture of the daily profile and allow one to minimize the time outside of normal levels. However, the majority of patients display low compliance to this instructed frequency of measurement, which has been attributed to inconvenience (pain, time, etc.). In fact, 60% of the patients that rely on insulin average less than a single measurement per day. That number increases to 95% for patients who do not use insulin.

In contrast to the finger-stick method employed in existing blood glucose meters, continuous glucose monitoring (CGM) uses an implanted sensor in the interstitial space that can automatically take hundreds of readings per day. Towards this end, different strategies have been developed for future sensors and there are several CGM devices that have recently been commercialized.

One type of glucose sensing assay that has been proposed for CGM is a competitive binding assay using the receptor Concanavalin A (ConA) and a competing ligand. ConA is a tetrameric protein at physiological pH, and has a primary binding site that binds to glucose and mannose residues with unmodified hydroxyls at 3, 4, and 6 positions on the ring structure. Each binding site on ConA is independent from the other monomers, and no cooperativity is seen between binding sites. The competing ligands that are used in these assays present glucose or mannose on their structure to allow for the competitive binding. When properly tuned, the population of ConA that is bound to the competing ligand at any given time significantly changes over physiological concentration ranges. Fluorescent labels have typically been introduced to each component to make this equilibrium binding capable of being interrogated by measuring changes in fluorescent properties.

One of the primary benefits of such an assay is its non-consuming nature. Commonly likened to immunoassays, this assay can be solely dependent on the concentration of glucose within the sample. Because the assay does not consume glucose, it is less dependent on the collective rates of diffusion, recognition, and consumption. For enzymatic based sensors, these rates must be balanced to have a stable test system and generate a repeatable response. Therefore, the biofouling that is commonly known to occur upon implantation can significantly change the test system of enzymatic sensors, resulting in significant error in glucose prediction over time. This can be seen in current commercially available enzymatic-based CGM devices that require many calibrations for accurate glucose concentration readings. Without these recalibrations, the changes in diffusivity from biofouling cause the glucose readings to have significant error. A working ConA-based assay could extend the lifetime of implantable sensors and/or decrease the number of recalibrations required to be confident in the outputted glucose reading because it is less sensitive to changes in diffusivity. Because the low patient compliance with finger-stick measurements is a primary reason for CGM devices, limiting the amount of required finger-stick recalibrations for a CGM technique would be ideal.

The ConA-based assay was first introduced using ConA and the polysaccharide dextran. Ever since the initial assay that was introduced, many different versions of this assay have been produced by changing the competing ligand, the transduction mechanism used, and the encapsulation method. However, while these assays have enormous potential as solution-based assays, they either report relatively low sensitivity to glucose or irreversibility problems. A major factor in the sub-optimal performance of existing ConA-based assays is the tendency for the reagents (e.g., competing ligand) to aggregate. As such, these assays have not yet been deemed effective for CGM because measured response can only be glucose-dependent for as long as the test system responds to glucose in a predictable way. Therefore, the underlying mechanism for the response must be repeatable during the working lifetime of the sensor.

This trade-off between sensitivity and repeatability has been slowly accepted as an inherent problem of ConA-based glucose sensing, decreasing the amount of attention directed its way. However, there has not been much work exploring the fundamental mechanism that explains these results. To date, most of the conclusions regarding the effectiveness of ConA-based glucose sensing stems from the measured responses from the collective assays published in the literature.

Despite the advances in glucose sensing assays noted above, a need exists for an improved glucose assay that demonstrates both sensitivity and reproducibility. The present invention seeks to fulfill this need and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure provides a ligand for Concanavalin A. The ligand comprises (a) a Concanavalin A binding component that binds to the primary glucose binding site and part or all of an extended binding site on Concanavalin A; and (b) a transduction component. The binding component is coupled to the transduction component, and the transduction component generates a detectable signal upon binding of the ligand to Concanavalin A. The binding component can be coupled directly or indirectly to the transduction component. The coupling can be a covalent bond, ionic bond, or any acceptable coupling in the art.

In some embodiments, the binding component comprises one or more mannose moieties. In some embodiments, the binding component comprises a trimannose moiety. In some embodiments, the trimannose moiety is 3,6-Di-O-(α-D-mannopyranosyl)-D-mannopyranose. In some embodiments, the binding component comprises a bimannose moiety. In some embodiments, the bimannose moiety is 6-O-α-D-mannopyranosyl-D-mannopyranose or 3-O-α-D-mannopyranosyl-D-mannopyranose. In some embodiments, the ligand has a binding affinity for Concanavalin A from about 10,000 to about 10,000,000 L/mol.

In some embodiments, the transduction component is a fluorophore, a Raman reporter, or a nanoparticle, or is electrochemically active. In some embodiments, the transduction component generates a detectable signal by a transduction mechanism selected from fluorescence intensity, fluorescent resonance energy transfer (FRET), fluorescence anisotropy, fluorescence lifetime, Raman spectroscopy, and metal enhanced plasmonics.

In some embodiments, the ligand further comprises a tether point for immobilization of the ligand to a structure or surface.

In some embodiments, the ligand further comprises a proteinaceous scaffold. In some embodiments, the binding component or the transduction component is coupled (e.g., covalently) to the proteinaceous scaffold, or both the binding component and the transduction component are independently coupled (e.g., covalently) to the proteinaceous scaffold. In some embodiments, the proteinaceous scaffold has a net negative charge. In some embodiments, the proteinaceoius scaffold is at least 20 kDa, 25 kDa, 30 kDa, 35 kDa, 40 kDa, 45 kDa, 50 kDa, or more. In some embodiments, the proteinaceous scaffold is or comprises ovalbumin or any derivative thereof. In some embodiments, the proteinaceous scaffold is or comprises RNase B or any derivative thereof.

In another aspect, the present disclosure provides a method for monitoring glucose in a sample. The method comprises detecting the competitive binding of a ligand to Concanavalin A in the sample, wherein the ligand has an affinity toward the primary glucose binding site and at least a portion of an extended binding site of Concanavalin A, wherein the ligand competes with glucose for binding to the primary binding site of Concanavalin A, and wherein a detectable signal is provided by a transduction component upon binding of the ligand to Concanavalin A.

In some embodiments, the detecting step comprises contacting the sample with the Concanavalin A and the ligand. In some embodiments, the equilibrium binding of the ligand to Concanavalin A is inversely related to the glucose concentration in the sample. In some embodiments, the sample is an in vitro or in vivo biological sample. In some embodiments, the biological sample is blood, blood plasma, blood serum, extracellular fluid, interstitial fluid, or aqueous humor fluid. In some embodiments, the detecting is performed in a continuous glucose monitoring assay. In some embodiments, the ligand comprises the transduction component. In some embodiments, the Concanavalin A comprises the transduction component. The ligand can be any ligand described herein. In some embodiments, each of the ligand and the Concanavalin A comprises a transduction component. In some embodiments, the transduction component of the ligand and the transduction component of the Concanavalin A are capable of mutually interacting as a FRET pair.

In another aspect, the disclosure provides a glucose monitoring system. The system comprises: (a) Concanavalin A; (b) a ligand having an affinity toward the primary binding site and all or part of the extended binding site of Concanavalin A, wherein the ligand effectively competes with glucose for binding to Concanavalin A; and (c) a transduction component to signal the state of assay binding.

In some embodiments, the ligand comprises the transduction component. In some embodiments, the ligand can be any ligand described herein. In some embodiments, each of the ligand and the Concanavalin A comprises a transduction component. In some embodiments, the transduction component of the ligand and the transduction component of the Concanavalin A are capable of mutually interacting as a FRET pair. In some embodiments, the system is adapted to an implanted biosensor. In some embodiments, the system is adapted to a subcutaneous biosensor.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates the recognition mechanism of a tuned system (Ka) using a multivalent competing ligand. Over time, increases in affinity due to aggregation (10*Ka, 100*Ka, 1000*Ka) change the competitive binding response due to the same glucose concentrations (% CLB: % competing ligand bound);

FIG. 2 compares expected fluorescence anisotropy response between 0 mg/dL and 300 mg/dL glucose for monovalent and multivalent assays over time (accounting for increases in affinity over time);

FIG. 3 illustrates a comparison between carbohydrate binding to the primary binding site of ConA and the extended binding site of ConA, using the crystal structure from Naismith, J. H., et al., Acta Crystallographica Section D: Biological Crystallography 50:847 (1994), incorporated herein by reference in its entirety. The black dots represent the amino acids capable of forming of hydrogen bonds to hydroxyl groups on the sugar (within 3.5 A of each other);

FIG. 4 compares the average particle size in solutions of ConA and various high-affinity ligands. The error bars indicate the standard deviation of 3 different samples;

FIG. 5 illustrates the synthesis scheme of APTS-MT via reductive amination to form a ligand with a single fluorophore and a single trimannose moiety;

FIG. 6A and FIG. 6B illustrate the fluorescent properties of APTS (6A) and APTS-MT (6B). Note that the excitation and emission shifts approximately 20 nm;

FIG. 7 illustrates the expected steady-state anisotropy of the bound (dark solid) and free (dotted) APTS-MT for a range of fluorescence lifetimes. The difference of the bound and free is indicated in the light bell-curve, and the APTS-lifetime is shown with the arrow;

FIG. 8 shows the fluorescence anisotropy responses of the 200 nM APTS-MT, 1 μM ConA assay for methyl mannose (circles), glucose (squares), and galactose (triangles);

FIG. 9 illustrates the predicted glucose vs. actual glucose for the FA competitive binding assay using 200 nM APTS-MT and 1 μM ConA;

FIG. 10 illustrates the excitation/emission spectra of APTS-MT and the TRITC-ConA used in the FRET assay. The dashed, horizontal shading lines indicate the spectral overlap, which is required for energy transfer;

FIG. 11A illustrates the fluorescence response to increasing concentrations of methyl α-mannose in the FRET assay (100 nM APTS-mannotetraose and 1 μM TRITC-ConA). The left vertical and right vertical lines are at 520 nm and 600 nm, respectively;

FIG. 11B illustrates the fluorescence intensity ratio at 520 and 600 nm as a function of monosaccharide concentration (methyl α-mannose and glucose);

FIG. 12 illustrates the predicted glucose vs. actual glucose for the FRET competitive binding assay using 100 nM APTS-MT and 1 μM TRITC-ConA;

FIG. 13 illustrates the excitation/emission spectra of ADOTA+ and AF647 in TRIS. The dashed, horizontal shading lines indicate the spectral overlap, which is required for energy transfer;

FIG. 14A and FIG. 14B illustrate the glucose response (with increasing physiologically relevant glucose concentrations) of a fluorescence assay comprising 500 nM ADOTA-OVA and 1 μM AF647-ConA. Specifically illustrated are the fluorescence intensities of the various glucose concentrations over a range of wavelengths (14A) and the normalized peak wavelength ratios for increasing glucose concentrations (14B); and

FIG. 15 illustrates the predicted glucose vs. actual glucose for the FRET competitive binding assay using 500 nM ADOTA-OVA and 4 μM AF647-ConA tracking fluorescence lifetimes.

### DETAILED DESCRIPTION

The present invention provides a competitive binding assay based on the protein Concanavalin A (ConA) and competing ligands that binds to the primary and part or all of the extended binding sites of Concanavalin A. The competitive binding assay and related compositions and methods are useful for glucose monitoring (e.g., continuous glucose monitoring (CGM)).

Improved continuous glucose monitoring devices have the potential to increase patient compliance and improve the management of diabetes. However, to date, assay approaches based on ConA have continually shown problems with sensitivity, stability, and reversibility in free solution. The present disclosure is based on the inventors' work in identifying problems with the existing ConA-based glucose detection assays, and rationally designing improved competing ligands for ConA-based assays.

Briefly, as described in more detail in Example 1, the inventors modeled the recognition and transduction mechanisms of the ConA-based assay in an attempt to identify desirable qualities to achieve an optimized assay. The models were used to explain the problems with previous ConA-based approaches. Briefly, monovalent ligands avoid aggregation/precipitation issues and, thus, provide constant affinities over time. However, such ligands have low affinity and, thus, low sensitivity for a detection assay. Typical multivalent ligands have increased affinity, but also aggregate over time, thus decreasing sensitivity over time. The models were also validated with experimental data, and used to optimize a ConA-based assay to track glucose concentrations with anisotropy.

As described in more detail in Example 2, the inventors identified the core trimannose of N-linked glycans as a high-affinity ligand that achieved the required affinities without leading to the aggregation that has caused previous assays to fail. Accordingly, a rationally designed fluorescent ligand was generated based on this core trimannose to achieve the desired qualities as defined by the previous models. The novel ligand was used in a ConA-based assay to track glucose concentrations with anisotropy. Furthermore, as described in more detail in Example 3, the novel ligand approach was further modified to incorporate Förster Resonance Energy Transfer (FRET). Finally, as described in more detail in Example 4, a second generation of the rationally designed fluorescent ligand was generated by fluorescently labeling a scaffold protein, which was further modified to display an N-linked glycan. The ligand based on a scaffold protein, in this case ovalbumin (OVA), was used in a ConA-based assay in an attempt to generate a cost-effective, rationally designed fluorescent ligand that could be encapsulated with a size exclusion membrane. This work establishes that the rationally designed fluorescent ligand concept can overcome the problems of previous ConA-based assays, and such an assay is expected to be capable of translation to practical applications.

Accordingly, the present invention provides a competitive binding (CB) assay for ConA that is both sensitive and reproducible. A key aspect of the assay is the use of a competing ligand that (1) has a high affinity toward ConA, which imparts sensitivity to the assay, and (2) is non-aggregating, which imparts a constant glucose response over time (reproducibility) to the assay.

In one aspect, the invention provides a ligand for Concanavalin A. The ligand is useful for competitive binding assays based binding to the lectin Concanavalin A (ConA), a representative sequence for which is set forth as SEQ ID NO:1. The ligand is useful for determining the levels of glucose in a sample, for example, by a continuous glucose monitoring (CGM) assay, by virtue of its competitive binding to the primary glucose binding site of ConA. The ligand is compatible with transduction and encapsulation techniques known in the art for glucose monitoring. In such glucose detecting and monitoring assays and systems, the ligand is a competing ligand and competes with glucose for binding to ConA.

In one embodiment, the ligand is a glycoconjugate that includes a ConA binding component and a reporting component. Unlike competing ligands in the literature that bind only to the primary binding site of ConA, the binding component of the disclosed ligand takes advantage of the extended binding site present on ConA, and presents structures that can be recognized and bound simultaneously by the primary and extended binding sites on a single ConA monomer. This full binding site has been identified to recognize specific mannose bearing structures found in N-linked glycans.

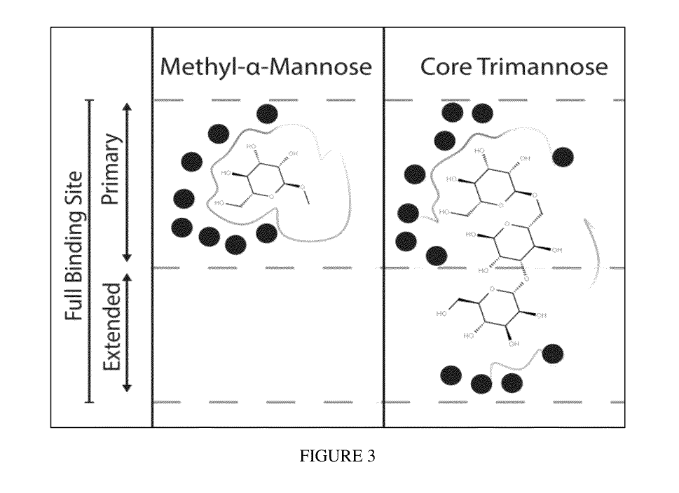

In certain embodiments, the binding component comprises one or more mannose moieties. In some embodiments, the binding component comprises a trimannose or bimannose moiety. In some further embodiments, the trimannose moiety comprises the fully recognized 3,6-di-O-(α-D-mannopyranosyl)-D-mannopyranose, or derivatives thereof. These derivatives include the bimannose “arms” of the core trimannose: 6-O-α-D-mannopyranosyl-D-mannopyranose and 3-O-α-D-mannopyranosyl-D-mannopyranose, herein identified as “6α-bimannose” and “3α-bimannose”, respectively. See FIG. 3, which shows the interaction of trimannose with the residues that compose the primary and extended binding sites of ConA, as opposed to glucose, which only interacts with the amino acids of the primary binding site.

In other embodiments, the binding component is a synthetic analog of these structures that presents structures that can be simultaneously recognized by the primary and extended binding sites of ConA. Because glucose is recognized by a portion of the full binding site (i.e., the primary binding site), the competing ligand can be in direct competition with glucose for ConA binding sites. Glucose should not bind to a ConA binding site that is bound to the aforementioned competing ligand. In addition, the aforementioned competing ligand should not bind to a ConA binding site when glucose is bound. FIG. 3 shows the interaction of glucose with the residues that compose the primary glucose binding site of ConA (including amino acids Tyr-12, Asn-14, Gly-98, Leu-99, Tyr-100, Ala-207, Asp-208, Gly-227, and/or Arg-228, or any combination or subcombination thereof). Note that these residues, in addition to the amino acid residues of the extended binding site (including amino acids Pro-13, Thr-15, and/or Asp-16, or any combination or subcombination thereof), comprise part of the full binding site that recognizes the ligands based on the bimannose moieties, trimannose moieties, or analogs thereof, as described herein.

The ligand has a high affinity toward ConA that allows the assay to be optimized to show the full sensitivity with regard to glucose recognition. The binding affinity of ConA to this ligand can range from 1.0*10⁴ to 1.0*10⁷ depending on the derivative of the binding component and the reporting component that is used. For example, the trimannose unit alone has previously been shown an affinity to ConA of ˜3.3*10⁵ M⁻¹.

The transduction component can be, but is not limited to, a fluorophore, quantum dot, nanodiamond, a Raman reporter, or a nanoparticle, that changes a measurable characteristic(s) between free and bound (by ConA) states. In other embodiments, the transduction component is not necessarily used in optical detection, but instead is electrochemically active. In any event, by virtue of the measurable characteristic, the binding of the competing ligand to ConA can be detected, and in some embodiments the levels of glucose present can be inferred. The transduction component can produce the change in a measureable characteristic by any mechanism known for producing detectable signals, such as by fluorescence intensity, fluorescent resonance energy transfer (FRET), fluorescence anisotropy, fluorescence lifetime, Raman spectroscopy, metal enhanced plasmonics, and the like. Any appropriate transduction can be incorporated into the ligand according to ordinary knowledge and skill in the art. For example, any known fluorescent moiety that provides changes to a measurable characteristic(s) between free and bound (by ConA) states can be used as the transduction component. An exemplary fluorescent moiety that can function in both FRET and anisotropy is ADOTA and related compounds, described in more detail below and in U.S. Patent Application Publication No. 2006/0211792, incorporated herein by reference in its entirety.

In some embodiments of the related methods and systems, described below, the ConA receptor comprises a transduction component. In some embodiments, the transduction component on the ConA is in addition to the transduction component of the ligand. In other embodiments, the transduction component on the ConA is in lieu of a transduction component on the ligand (i.e., the ligand does not comprise a transduction component).

The transduction component can be tethered to the ConA binding component in different ways. In certain embodiments, this tethering can be performed via reductive amination. In this scheme, an amine-bearing reporter molecule can be tethered to reducing terminus of a sugar-structure. An amine-bearing reporting component can form a Schiff base with the carbonyl group on the open-chain sugar. This Schiff base can be reduced into a stable bond with a reducing agent such as sodium cyanoborohydride. The pure glycoconjugate can be separated from the original reagents to be used. This scheme destroys the structure of the sugar at the reducing terminus by forcing it to stay in open-chain form. Therefore, a sugar with an additional sugar attached to the ConA binding component must be used to display the ConA binding component appropriately. For example, to present trimannose as the ConA binding component of a ligand tethered by reductive amination, mannotetraose is used as the initial sugar structure.

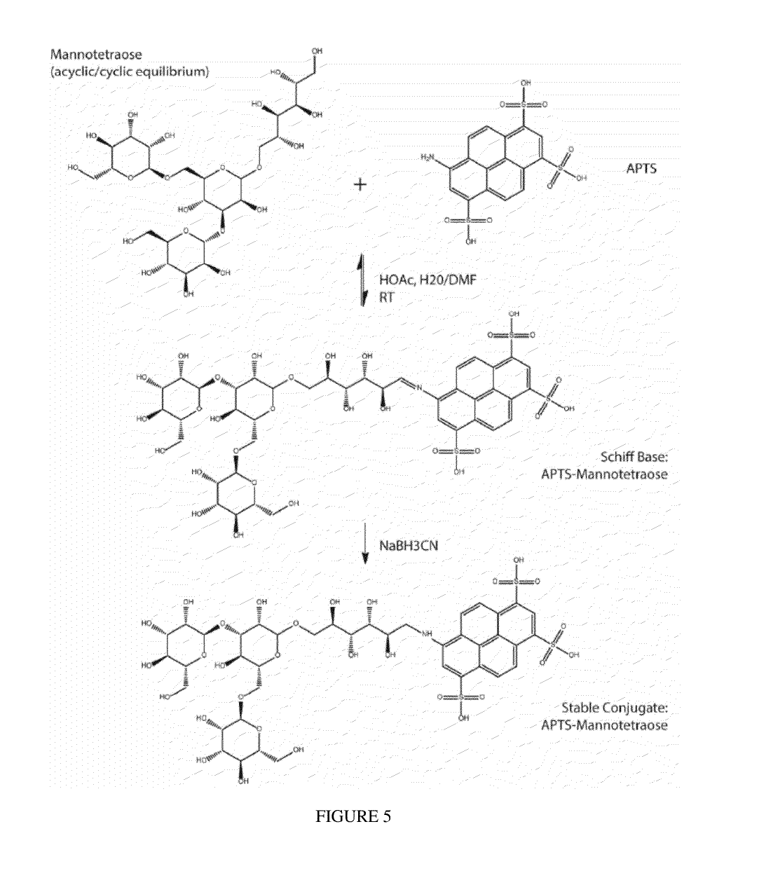

A representative ligand (a trimannose-bearing glycoconjugate with an APTS fluorophore) of the invention is shown at the bottom of FIG. 5, which illustrates a schematic for tethering via reductive amination. The representative glycoconjugate is prepared by Schiff base reduction from an open-chain mannotetraose and an amine-bearing fluorophore (e.g., 8-amionopyrene-1,3,6-trisulfonic acid (APTS)). Reaction of the open-chain mannotetraose and amine-bearing fluorophore provide a Schiff base, which is reduced to provide the fluorescent glycoconjugate product. The APTS-trimannose interacts with the residues that compose the full binding site of ConA.

In other embodiments, glycosylhydrazides can be used to tether the components. A hydrazide-bearing reporter molecule can form a Schiff base with the carbonyl group on the open chain of the sugar, and the ring is allowed to reform without reduction. This glycosylhydrazide has been shown to be very stable. This allows the ConA binding component to be used as the initial sugar-structure. For example, to present trimannose as the ConA binding component of a ligand tethered with glycosylhydrazides, trimannose is used as the initial sugar structure.

It will be appreciated that the structure of the ligand can be further modified to include, for example, tether points from which additional functionality can be added to the ligand or through which the ligand can be immobilized to a structure and/or surface. This can be done by using reductive amination to tether a heterofunctional crosslinker to the reducing terminus of the ConA binding component.

Accordingly, the ligand can have the transduction component coupled to the transduction component, directly or indirectly. The coupling can be any coupling mechanism/structure known in the art, such as an ionic coupling, a covalent coupling, and the like. In some embodiments, a recognition component can be coupled to the transduction component.

In some embodiments, the ligand comprises a scaffold structure on which the other components are tethered (e.g., covalently coupled). In some embodiments, the scaffold is a proteinaceous scaffold. For example, the structure can be a protein. It will be appreciated that the protein scaffold is not limited to any specific protein. Persons of skill in the art can readily determine the appropriateness of a candidate protein for the scaffold.

The protein can be of any appropriate size that can be readily determined by persons of skill in the art based on various factors. For instance, a scaffold can help prevent leaching of the ligand from an implantable monitoring device. To prevent leaching, the scaffold provides bulk to the ligand such that it cannot easily penetrate the structure (e.g., semi-porous membrane) of the device. The size of the scaffold can also be adjusted to ensure specific signaling capacities for any transduction component attached thereto in consideration of the transduction mechanism to be implemented by the component. In some embodiments, the scaffold is, for example, at least 20 kDa, 25 kDa, 30 kDa, 35 kDa, 40 kDa, 45 kDa, 50 kDa, or more, or any sub-range therein.

In some embodiments, the scaffold has a net negative charge, which can be useful when using native ConA receptor or most modified ConA receptors, which are also negatively charged. This attribute of the ligand would help avoid non-specific electrostatic binding. In other embodiments, the net charge of the proteinaceous scaffold is adjusted to help prevent leaching through a membrane, such as a semi-porous membrane of an implantable device. In this regard, the charge can be adjusted to promote electrostatic repulsion from the membrane to prevent leaching.

In some embodiments, the scaffold is selected to facilitate the ease of generating the final ligand, (e.g., to facilitate ease of tethering other components such as the binding component or transduction component). For example, in some embodiments, the protein scaffold comprises a single glycosylation site, e.g., the protein scaffold has a sequence capable of supporting a single N-linked glycan for the coupling of a component such as the ConA binding component. N-linked glycans are typically attached to proteins at the nitrogen in the side chain of asparagine (Asp) in a consecutive sequence of amino acids. The sequence is typically Asn-X-Ser or Asn-X-Thr sequence, where X is any amino acid except proline. The glycan will typically be composed of N-acetyl galactosamine, galactose, neuraminic acid, N-acetylglucosamine, fructose, mannose, fucose, and other monosaccharides.

In one illustrative embodiment, the scaffold is or comprises ovalbumin. As described below, ovalbumin provides the advantage of size (˜45 kDa), of having a single glycosylation site that can present a multiple-mannose binding component (e.g., Asp-292), and multiple lysine residues to enable the tethering of a transduction component. Thus, the use of the term ovalbumin in the context of a ligand of the present invention includes a wild-type ovalbumin protein (a representative sequence is set forth in SEQ ID NO:2), or any derivative thereof. Derivatives include any appropriate subdomain(s) thereof and/or sequence variants (such as with 50%, 55%, 60%, 65%, 70%, 75%, 80%, 85%, 90%, 95%, 99% sequence identity thereto), which can be determined by those skilled in the art. As described below, one advantage of the present disclosure, as illustrated with the use of ovalbumin, is that the scaffold can be used to rationally design and generate a competing ligand that displays a transduction element at a sufficiently close proximity to a FRET partner on the ConA receptor to provide a detectable signal upon binding. Thus, the present disclosure also encompasses ligands with ovalbumin derivatives that have been modified to facilitate this function. For example, portions of the wild-type ovalbumin can be omitted that do not affect the glycosylation site for the binding component (e.g., trimannnose moiety) and at least one attachment site for a transduction component (e.g., a lysine residue). In other embodiments, the derivative can be a mutant that has one or more of the lysine residues substituted or deleted such that only a limited number of the original lysine residues are available for attachment of an exogenous moiety (e.g., a fluorescent transduction element). Finally, it will be appreciated that many other modifications can be readily made to generate derivatives that facilitate or permit the attachment of binding components or transduction components by appropriate and well-known protein chemistry approaches.

In another illustrative embodiment, the proteinaceous scaffold can be Ribonuclease B (RNase B). An exemplary full-length amino acid sequence for RNase B (bovine) is set forth in SEQ ID NO:3. As with the above-described example of ovalbumin, the use of the term RNase B includes derivatives thereof (e.g., sub-domains and/or sequence variants thereof). RNase B is a naturally-occurring glycoprotein that presents a single high-mannose N-linked glycan at Asp-34. It is a globular protein with a molecular weight of 15 kDa (˜4 nm in diameter). The native structure presents eleven primary amines (N-terminus and ten lysines), each of which could be used to conjugate commonly-used amine-reactive probes (via succinimidyl ester). The isoelectric point of native RNase B is 9.3, making it net positive at physiological pH. There are many well-known approaches to modify this isoelectric point, including the capping of the primary amines with uncharged/negative moieties. In fact, the fluorescent labeling of these primary amines would decrease the isoelectric point and can serve as a way to have a negatively charged fluorescent molecule at physiological pH. Derivatives can be rationally design according to the preferred characteristics of the scaffold. For example, the scaffold can comprise at least a subdomain of RNase B that retains the Asp-34 (the position number referring to the position in the full-length sequence) and at least one lysine residue. The remaining lysine residues can be modified (e.g., deleted or substituted) to facilitate the addition of a component on the remaining, preferred lysine residue(s).

It will be appreciated that the lysine residue, or indeed any intended target residue for the attachment of a transduction component to a scaffold such as RNase B, ovalbumin, and the like, can be rationally selected based on known folding configurations to display the transduction component at a sufficiently close proximity to the ConA receptor upon binding of the ligand. This rational configuration can facilitate FRET signaling when the ligand is bound to the ConA receptor.

In another aspect, the invention provides a method of detecting glucose, such as would be applied in a glucose sensing assay. In the method, the competitive binding of a ligand, as described herein, to Concanavalin A is detected in the sample. As described herein, the ligand has an affinity toward the primary glucose binding site and at least a portion of an extended binding site of Concanavalin A, wherein the ligand competes with glucose for binding to the primary binding site of Concanavalin A, and wherein a detectable signal is provided by a transduction component upon binding of the ligand to Concanavalin A.

By this method, glucose concentration is determined by assaying the competitive binding of the ligand of the disclosure to ConA. As more ligand is determined to be bound, there is an inferred lower level of glucose in the sample. In some embodiments, the detectable signal is compared to a reference standard that is associated with a known level (e.g., concentration) of glucose in a sample. Thus, the relative levels of ligand binding determined from the detectable signal can be used to infer the relative levels of glucose in the sample.

In some embodiments, the method comprises steps of providing a plurality of ConA receptors and a plurality of ligands, as disclosed herein, into the sample before detecting the competitive binding.

The methods can be incorporated into homogenous or heterogeneous assays. In some embodiments, the sample is in vitro or in vivo. In some embodiments, the sample is a biological sample, including for example, the biological sample is blood, blood plasma, blood serum, extracellular fluid, interstitial fluid, aqueous humor fluid, and the like, which typically have detectable levels of glucose that are informative for the health state of the source subject.

As indicated above, a representative sequence for a ConA monomer is set forth in SEQ ID NO:1. The ConA receptor can be native, succinylated, acetylated, PEGylated, or incorporate any other derivative or modification that allows the extended binding site to remain functional. When ConA exists in a tetrameric form, the glucose binding site in each ConA monomer is independent of the glucose binding sites in other monomers. Accordingly, in the scope of the present disclosure, ConA can be used in monomeric, dimeric, tetrameric, or greater forms, depending on the derivation method used. ConA can either be free or be immobilized to a structure and/or surface.

In certain embodiments, the binding of the ligand to ConA is determined by fluorescence polarization (anisotropy). Using fluorescence polarization/anisotropy, the fluorescent ligand is engineered to have a lifetime that is centered between the average rotational correlation lifetimes between the free and bound states and an intrinsic anisotropy nearing the maximum of 0.4. As described below in Example 2, the relationships of the fluorescence anisotropy sensitivity (change between free and bound to ConA states) for a competing ligand with a given molecular weight (MW) and fluorescence lifetime were modeled. One example, described herein, is a ligand that has trimannose as its ConA binding component and APTS as its reporting component. The intrinsic anisotropy of an APTS conjugated glycan using reductive amination is demonstrated. Such intrinsic anisotropy of APTS is ideal for such an assay. In addition, the time-resolved fluorescence lifetime of an APTS-conjugated glycan using reductive amination is determined to be about 5 ns.

The advantage of using a single presentation of a ligand that utilizes part of the extended binding site of ConA in addition to the primary binding site of ConA is shown below. This includes its ability to avoid the aggregation that is commonly seen for the traditional high-affinity competing ligands. The glucose response of a representative fluorescence anisotropy assay of the invention using APTS-MT and native ConA is described below. The affinity of this APTS-MT-to-native ConA (5.61*10⁶ M⁻¹) allowed for the ConA concentration in the assay to be well below its solubility limit (˜1 μM) and optimize the recognition mechanism of the assay.

In other embodiments, the binding of the ligand to ConA is determined via Förster Resonance Energy Transfer (FRET) as a transduction mechanism. In one FRET-based embodiment, the ligand comprises a transduction component that serves as the FRET donor and ConA is labeled with a FRET acceptor. In another embodiment, ConA is labeled with a FRET donor and the ligand contains a transduction component that serves as the FRET acceptor. Either the fluorescence lifetime or the fluorescence intensity can be monitored from the assay. The assay can be engineered to only induce FRET when the ligand and ConA are bound. As described in Example 4, the structure of a ligand presenting a single trimannose-bearing N-linked glycan via ovalbumin was modeled and engineered to present the fluorophore (e.g., ADOTA conjugated to glycated ovalbumin) within an appropriate Förster radius to produce a sufficient change in signal upon binding of the ligand to ConA. Thus, minimal FRET will occur when the sensing assay component bearing the FRET donor is free by limiting the concentrations of the sensing components and the fluorescence lifetime of the donor.

In another aspect, the disclosure provided a glucose monitoring system and/or a device that comprises ConA, a ligand having an affinity toward the primary binding site and all or part of the extended binding site of Concanavalin A, wherein the ligand effectively competes with glucose for binding to Concanavalin A, and a transduction component to signal the state of assay binding.

In some embodiments, the ligand comprises the transduction component. In some embodiments, the ligand is any ligand described hereinabove. In other embodiments, the ligand does not comprise a transduction component, but rather the ConA comprises the transduction component. In some embodiments, both the ConA and the ligand comprise a transduction component such that they are capable of cooperating as a FRET pair (donor and acceptor) to provide a detectable signal upon binding of the ligand to the ConA, as described above.

The system can be adapted as part of an implantable biosensor, such as a biosensor that is implanted into the skin (a subcutaneous biosensor). In this regard, this sensing chemistry can be left in free solution by withholding it within a semi-permeable membrane that prevents leaching of components while allowing the free diffusion of glucose. This and other encapsulation technologies, such as use of polymeric microspheres and layer-by-layer (LbL) approaches, can be used to retain the ligands and ConA in place within the implanted region while allowing glucose to freely diffuse into the biosensor.

For example, sacrificial spherical templates (i.e., melamine formaldehyde and calcium carbonate) have been successfully loaded with proteins and sensing chemistry that can then be exposed to alternating layers of poly-electrolytes which coat the template. These cores can then be dissolved, freeing the chemistry within the micron-sized LbL capsule that can then serve as a semi-permeable membrane for sensing purposes. Advantages of this technique include the fine level of control for the mesh size of the capsule and the high synthetic reproducibility.

Alternatively, sensing chemistries have been embedded within a dense matrix in an attempt to maintain long-term functionality. Poly-(ethylene glycol) (PEG) has been employed in this manner due to its proven biocompatibility and hydrophilic nature. Microspheres can be created using PEG by crosslinking the individual chains via thermo-chemical or photo-chemical initiation, resulting in a mesh of PEG chains in which chemistry can be embedded. The effective pore size of this mesh can be altered by varying the average molecular weight of the PEG and/or changing the water content within the precursor solution prior to crosslinking. With these variations, the mesh size for PEG can be tailored to be suitable for sensing purposes. For example, another encapsulation strategy involves combining a water-in-oil emulsion technique with the addition of sugar crystals to the precursor PEG solution to form assay-filled pores within the hydrogel matrix of the microspheres. This microporation technique has been shown to be functional with the ConA/Dextran glucose sensitive assay, displaying a reversible response over several days. Because microporated PEG spheres allowed competitive binding within the larger pores while providing for diffusion of smaller analytes due to the selectively permeable mesh, it is likely that similar biocompatible microporated microspheres can be viable candidates for housing the aggregative ConA/competitive ligand glucose sensing assay.

While the preferred embodiments of the invention has been illustrated and described, it will be appreciated that various changes can be made therein, including using quantum dots, Raman reporter molecules, nanodiamonds, or nanoparticles, or incorporating into various implantable device configurations, without departing from the spirit and scope of the invention.

The following examples are provided for the purpose of illustrating, not limiting, the material disclosed herein.

### EXAMPLES

### Example 1

### Mathematical Modeling of ConA-Based Glucose Sensor Based on Competitive Binding with Fluorescence Anisotropy

ConA-based assays have primarily displayed a lack of sensitivity or a lack of repeatability in their glucose response. The lack of sensitivity or a lack of repeatability in glucose response was explored by separating the measured glucose response into the recognition and transduction mechanisms.

The recognition responses were modeled for typical competing ligands/as says used in the literature, and combined with an optimized fluorescence approach to yield expected fluorescent glucose responses. Because aggregation is known to increase the apparent affinity between multivalent ligands and multivalent receptors, preliminary models were generated for assays that were initially optimized with multivalent ligands but increase in affinity over time. These models accurately predict the low sensitivity for monovalent ligands and the lack of repeatability in the responses with multivalent ligands as seen in the literature. This explains the aforementioned trade-off no matter the optical approach.

By separating the measured response of a general ConA-based sensing assay into its recognition and optical transduction mechanisms, and by using the explanation of the glycoside cluster effect, the shortcomings of traditionally published assays and the apparent trade-off between sensitivity and repeatability can be explained.

In order to separate the general glucose response of ConA-based sensing assay into its recognition and optical transduction mechanisms, anisotropy was used to transduce the binding equilibrium. A competing ligand was used that sufficiently increases its fluorescent anisotropy upon binding to ConA. Equation 1 describes the expected measured (rₜ) anisotropy signal according to the anisotropy of the bound (r_{b}) and free (r_{f}) competing ligand and the fluorescence intensity that comes from the bound (f_{b}) and free (f_{f}) competing ligand. The quantity fₜ is the total fluorescence intensity from the sample.

r_{t(glu)}fₜ=r_{b}f_{b(glu)}+r_{f}f_{f(glu)} (1)

Equation 2 shows that the recognition mechanism and the transduction mechanism must both be optimized to generate a sensitive assay. In Equation 2, the % CLB is the amount of the fluorescent competing ligand that is bound to ConA, which is equivalent to the fractional fluorescence intensity coming from the bound population if the quantum yield of the fluorescent ligand does not change upon binding to the receptor. G is the glucose concentration in the system. In this work, the aim was to identify the assay expected to maximize the change in the anisotropy in response to the glucose concentration changing from 0 mg/dL to 300 mg/dL. The (r_{b}-r_{f}) term of this assay is the change in anisotropy upon binding to ConA and is related to the transduction mechanism. The (Δ% CLB)/ΔG term is the change in the percent competing ligand that is bound for a given change in glucose concentrations and is related to the recognition mechanism.

$$\frac{Δ {r}_{t}}{Δ G} = ( {r}_{b} - {r}_{f} ) \frac{Δ \% CLB}{Δ G} \qquad ( 2 )$$

If both components are not optimized, the level of optimization of the measured response will be quite low.

By assuming the quantum yield and the radiative lifetime to remain unchanged upon binding, the percentage of the fluorescence coming from the bound and free competing ligand can be equal to the percentage of the competing ligand that is bound and free. These values can be modeled for a given set of association constants and concentrations of the components in the assay using the exact solution described in Wang, Z.-X., FEBS Letters 360(2):111-114 (1995), incorporated herein by reference in its entirety.

The glucose recognition mechanism for two distinct assays was modeled. A 2D sensitivity plot was generated to map the expected glucose-sensitivity for different ConA concentrations and association constants between the ConA and the affinity of the competing ligand for an expected competing ligand concentration of 500 nM (not shown). The first assay uses an association constant that is typically seen for monovalent sugars (Kₐ=10⁻³). The second assay uses an association constant that is typically seen for multivalent presentation on a dendrimer or nanoparticle based structure (Kₐ=10⁻⁶). Concentrations of each component in the competitive binding assays are used to maximize the binding while staying below the solubility limit of ConA.

Aggregation is typically observed in assays with multivalent receptors and multivalent ligands and occurs over time with a speed dependent on that specific assay. Upon aggregation, the apparent affinity between the receptor and ligands increases due to the chelation between molecules. For the competitive binding assay using the multivalent competing ligand, this increase in affinity was accounted for by increasing the affinity by a factor of 10, 100, and 1000. Because aggregation occurs over time, these affinities are intended to display the time-dependent trends as the aggregate changes binding mechanics over time. These glucose recognition mechanisms were then combined with a fluorescence transduction mechanism (r_{b}=0.35 and r_{f}=0.05) that was well optimized, and the expected responses were simulated for each assay.

Alternatively, the recognition mechanism of an assay can use a competing ligand that presents a single monosaccharide that binds to ConA. However, because of the low affinity of ligands with single monosaccharides and the solubility limit of ConA (about 100 uM), the ConA concentration cannot be high enough to sufficiently allow for significant binding between ConA and the competing ligand. Therefore, increasing glucose concentrations does not significantly change the % CLB (competing ligand bound). This conveys the primary reason that multivalent ligands are used as competitors: to increase the apparent association constant to optimize the recognition mechanism with lower assay concentrations.

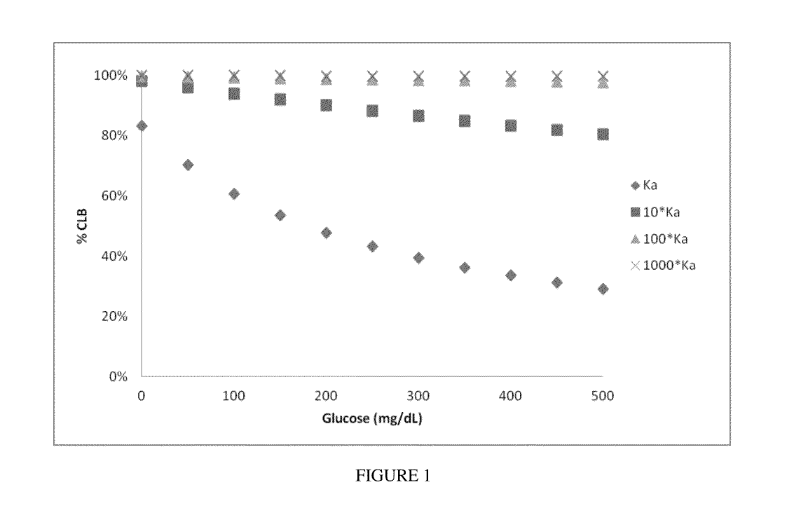

FIG. 1 illustrates the recognition mechanism of an assay that uses a competing ligand that presents multiple monosaccharides on its surface (such as a glycosylated dendrimer). The increased association constant allows the assay to be optimized (diamonds) and significantly respond to physiological glucose concentrations. However, as the affinity increases due to aggregation and chelation, the competitive binding shifts keeping ConA bound to the competing ligand over the same glucose range (squares, triangles, x's). Eventually, the assay does not respond to physiological glucose concentrations. This trend could be reversed, but would require significantly higher glucose concentrations than what is seen in the body to do so.

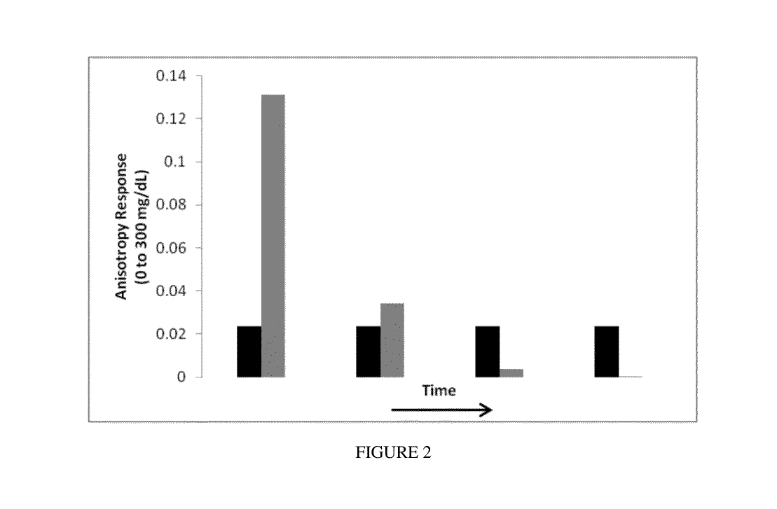

These results for the recognition mechanism using the monovalent and multivalent competing ligand were combined with an ideal fluorescence transduction mechanism to show what one would measure for the different assays. The fluorescence mechanism showed an increase in the anisotropy of the fluorescent competing ligand from 0.05 to 0.35 upon binding to ConA. FIG. 2 shows the responses for the monovalent ligand (black) and the multivalent ligand (gray) using Equation 1.

Because the monovalent ligand does not allow for extended aggregation/precipitation between multiple components, the affinity stays constant over time and the resulting anisotropy response does as well. However, because the affinity is weak, the sensitivity of the response is low because it is not capable of being properly tuned. On the other hand, the increased affinity of the multivalent originally allows the assay to be properly tuned, as seen by the very sensitive anisotropy response (gray). However, the multivalent presentation of monosaccharides capable of binding to the primary binding site of ConA allows time-dependent aggregation that increases the apparent affinity of the ConA-CL interaction. This increase in affinity increases the concentrations of glucose required to induce competitive binding—decreasing its sensitivity to physiological glucose concentrations over time. Therefore, this displays the apparent trade-off shown in literature between sensitivity and repeatability in ConA-based competitive binding assays.

Using monovalent and multivalent competing ligands, glucose recognition curves show for each ligand according to their affinity constant to ConA. Monovalent competing ligands show a non-optimized recognition mechanism, but the glucose response is constant over time. On the other hand, multivalent competing ligands allow for high initial sensitivities due to optimized recognition mechanisms, but aggregation-induced increases in affinity display changes in the fluorescent response over time. These responses are typical of ConA-based assays published in the literature. To avoid this trade-off in free solution and display the full potential of such an assay, an improved assay must instead generate higher affinities while preventing disadvantageous aggregation over time.

The challenges presented to achieve an improved assay are numerous. Even if aggregation is somehow avoided, the traditional type of fluorescent competing ligand is not ideal for the transduction mechanism of an anisotropy-based assay because high apparent affinity is typically connected with high molecular weight. This is exemplified with dextran, the most commonly used competing ligand in such assays, which is a branching polymer built of glucose subunits. As the molecular weight of dextran increases, it presents a higher number of termini that ConA can bind which increases the apparent affinity. One commonly used fluorescent ligand in ConA-based assays is 70 kDa FITC-dextran, but this ligand still only displays an apparent affinity of ˜15,000 M⁻¹ to ConA. According to a 2D sensitivity map for the transduction mechanism of a fluorescence anisotropy assay, the competing ligand's molecular weight needs to be closer to 1 kDa than 100 kDa. Ultimately, the ideal characteristics of the fluorescent ligand with regard to the transduction mechanism would be a MW of ˜1 kDa and a fluorescence lifetime of ˜5 ns.

The traditional type of competing ligand is also not ideal for assays that employ distance-dependent transduction mechanisms, like FRET. These traditional, multivalent competing ligands typically show low-efficiencies of FRET-transfer upon binding because they allow for large distances between the donor and acceptor fluorophores upon binding. For example, a 70 kDa fluorescent dextran that is ˜10 nm in diameter (a common ligand in traditional ConA-based glucose sensing assays) allows ConA to bind to a terminal glucose group that is 10 nm away from the fluorophore on the dextran. In addition, the corresponding fluorophore of the FRET-pair on ConA can be several nm away from this binding site. Therefore, this binding event cannot be tracked with fluorescence because efficient transfer typically requires the FRET-pair to be within 5-6 nm of each other. The invention of this disclosure allows for improved efficiency of distance-dependent transduction mechanisms like FRET.

In summary, this Example described models of the recognition and transduction mechanisms of the ConA-based competitive binding assay. These models were validated with experimental results with 4 kDa FITC-dextran as the fluorescent ligand. An assay based on this ligand was optimized and developed into a fluorescence anisotropy sensor for glucose concentrations.

### Example 2

### Rationally Designed Fluorescent Ligands for ConA-Based Anisotropy Assays

### Introduction

In Example 1, the requirements were elucidated for a dynamic competitive binding assay in which the fluorescence of the competing ligand is tracked with anisotropy. Briefly, this required the competing ligand to have an affinity that is higher than what is typically seen for monosaccharides. To date, this has been achieved by implementing fluorescent ligands that presented multiple monosaccharides on a single ligand (e.g., dextrans & dendrimers). While multivalent presentation is known to increase the apparent affinity to a receptor through proximity effects, problems with aggregation due to extensive crosslinking result in the lack of reversibility of the assay in free solution. This has been considered the major weakness of this type of assay. Thus, there has been a tradeoff between long-term repeatability/reversibility and initial sensitivity in free solution.

In this Example, a rationally designed fluorescent ligand is described, which is engineered to display the ideal properties for the recognition and transduction mechanisms of a ConA-based assay. This ligand displays the unique capability to achieve the required affinity without allowing for aggregation, and it can be effectively inhibited with glucose and mannose. This rationally designed fluorescent ligand is synthesized, characterized, and implemented into an anisotropy based assay for glucose sensing.

### Results/Methods/Discussion

To achieve increased binding affinities of ligands to ConA, the present inventors addressed potential binding of the ligands to subsites of the lectin. This approach has an extended binding site near the primary binding site on the lectin for specific sugar moieties. Because additional interactions are made between the sugar and the full binding site, the affinity is increased.

Adjacent to ConA's primary binding site is an extended pocket that has been shown to form additional hydrogen bonds to the core trimannose of N-linked glycans (trimannose). X-ray crystallography data was obtained for ConA's interaction with methyl-alpha-mannose and the core trimannose (1CVN and 5 CAN), and the relative orientations of the ConA's binding to each ligand was determined using the Ligand-Interaction tool in the Maestro software (v. 9.5). FIG. 3 shows the amino acids (black dots) in the crystallography structure that are within a distance that is capable of forming hydrogen bonds. These amino acids display the extended binding site to which the core trimannose binds. This shows that ConA forms hydrogen bonds with each mannose group of the trimannose, which should increase the affinity without leaving moieties for additional ConA to bind. It also shows that the trimannose could compete with the monosaccharide for binding to a ConA subunit because they both bind to the primary binding site.

Aggregation Studies

To explore whether the interaction of ConA with the core trimannose leads to aggregation in free solution, dynamic light scattering (DLS) measurements were performed. This interaction was compared to the interactions between ConA and other high-affinity ligands that are commonly used in ConA-based assays. Dextran, dendrimer and trimannose were added to separate cuvettes of ConA solutions in TRIS buffer, and allowed to interact for 12 hours at 22° C. The TRIS buffer was 10 mM TRIS, 1 M NaCl, 1 mM CaCl₂, 1 mM MnCl₂, at pH 7.4. The dextran was 2 MDa and used as received from Sigma. The dendrimer was a generation three glycosylated dendrimer (24 terminal amines, 24 terminal glucose residues).

Dynamic light scattering was then used to examine the average size of particles formed in comparable solutions. As controls, solutions of the individual components were also scanned to determine the particle size in the absence of aggregation. The final concentration was 3 μM for the competing ligand and 3 μM for ConA. Negative controls were run for each competing ligand (3 μM) without ConA present. ConA was also independently added to a separate control cuvette (3 μM). All solutions were in TRIS buffer and were filtered prior to combining the solutions. After 12 hours, the solutions were mixed to allow for the re-suspension of any settled material and dynamic light scattering studies were performed. The reported data is the average particle size as determined by percent-volume.

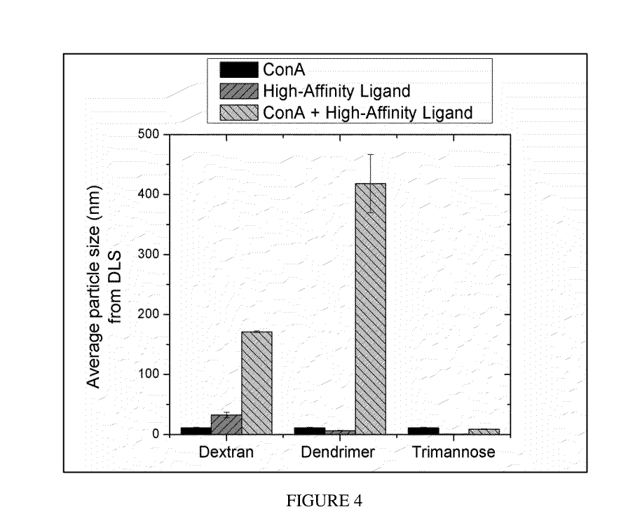

FIG. 4 shows that the average size of the particles is much larger after 12 hours for solutions of ConA paired with dendrimer and dextran, indicating that ConA and the high-affinity ligands have already formed extensive aggregates. In comparison, the average size of particles in the solution that contains trimannose and ConA did not increased in size, indicating that, if binding occurred, a single presentation of trimannose significantly decreases the extent of aggregation and potentially prevents it completely.

Generation of Fluorescent Ligands

Having demonstrated that a single trimannose moiety does not lead to aggregation, a rationally designed ligand was generated where a fluorescent ligand that has a single fluorophore and displays a single trimannose moiety. Therefore, the commonly used periodate oxidation method to generate carbonyl groups on a polysaccharide for fluorophore attachment was not appropriate because 1) it would destroy the ring mannose moieties required for binding to ConA's full binding site, and 2) it could introduce several fluorophores to a single glycan.

Reductive amination is a more controlled method that has been used to label glycans for subsequent chromatographic or electrophoretic separation and identification of the glycans. See, e.g., Bigge, J. C., et al., Analytical Biochemistry 230:229-238 (1995) and Guttman, A., et al., Analytical Biochemistry 233:234-242 (1996), each of which are hereby incorporated herein by reference in their entireties. By using an amine-bearing fluorophore, this method introduces a single label at the reducing termini of the glycan. This causes the reducing sugar of the glycan to be acyclic but leaves the remaining sugars of the glycan in their cyclic, unaltered form to be recognized by receptors. Thus, the trimannose bearing mannotetraose was used to maintain the trimannose structure after conjugation.

8-Aminopyrene-1,3,6-trisulfonic acid (APTS) was chosen as the amine-bearing fluorophore to attach to the mannotetraose. APTS is a water-soluble fluorophore whose fluorescence is independent of pH over a wide range. It has been used to label glycans enzymatically cleaved from glycoproteins via reductive amination to facilitate their separation via capillary electrophoresis due to its three negative charges per fluorophore at neutral pH. Because ConA's isoelectric point is around 5, these negative charges also minimize non-specific electrostatic interactions between the fluorescent ligand and ConA at physiological pH.

Mannotetraose was purchased from Dextra Laboratories. Mannotetraose (0.9 mg, 1.35×10⁻⁶ mol) was mixed with 13.5 μL of 1 M APTS (1.35×10⁻⁵ mol) prepared in 15% acetic acid aqueous solution. Then, 54 μL of 15% acetic acid aqueous solution was also added into the mixture. The acidic environment promotes the ring-opening of the reducing terminus of mannotetraose. The reaction mixture was stirred for 5 minutes at room temperature. Then, 13.5 μL of 1 M sodium cyanoborohydride (NaBH₃CN, 1.35×10⁻⁵ mol) in THF was added into the reaction and had it stirred for 14 hours at room temperature. The schematic is shown in FIG. 5. Sodium cyanoborohydride is a reducing agent that converts the Schiff base into the stable APTS-mannotetraose (APTS-MT) conjugate. APTS-glucose and APTS-maltotriose were also synthesized using this method.

It is noted that 2-picoline borane is another effective reducing agent for the reductive amination of glycans with fluorophores. Furthermore, it is noted that the above-described type of reaction can be performed to conjugate the glycan to other fluorophores as well. Most often, these fluorophores have an aromatic amine to maximize the efficiency of the reaction over a shorter time. These aromatic amines can be deprotonated even under the acidic conditions for the reductive amination. Fluorophores have been used that present hydrazides and thiosemicarbazides in non-reducing conditions. This allows the reducing sugar to return to the cyclic form and still be attached via the hydrazide. This is more stable than the Schiff base, but it is not as stable as the conjugate that has been reduced.

Separation of the Labeled Ligand with HILIC Chromatography

Following the reductive amination synthesis, the crude product was purified by hydrophilic interaction liquid chromatography (HILIC) and identified via its change in absorbance. An HPLC system containing a solvent delivery module Model 126, an auto injector Model 508, and a photodiode array detector Model 168 operating under 32 Karat software control (Beckman-Coulter, Fullerton, Calif.) was used for reaction monitoring. HILIC separations were obtained on a 4.6 mm I.D., 150 mm long analytical column packed with a 3 μm HILIC stationary phase (Phenomenex, Torrance, Calif.). Solvents used were A: 100% water; B: 5% water, 95% acetonitrile both containing 10 mM ammonium formate, 5 mM formic acid, pH 9. Isocratic: 80% B, 10 min.

Confirmation with Mass Spectrometry

The postulated structure of the product was confirmed using electrospray ionization mass spectrometry in negative ion mode. Mass spectra were acquired in negative ion mode using an MDS SCIEX (Concord, Ontario, Canada) API QStar Pulsar. Sample was dissolved in (methanol) and electrosprayed using ionspray (needle) at −4.5 kV. Sheath gas and curtain gas flow rates were set to 40 and 20 psi, respectively. The sample flow rate was 7 μl/min. Multiply charged ions were detected.

Fluorescence Characterization of APTS-MT—Excitation/Emission/Intrinsic Anisotropy

The final concentrations of the APTS-MT and APTS-glucose were estimated using absorbance spectroscopy with the extinction coefficients found in the literature for APTS, and assuming the extinction coefficients at the maximum absorbance to be equivalent. See Reeves, P. J., et al., Proceedings of the National Academy of Sciences of the United States of America 99:13419-13424 (2002), incorporated herein by reference in its entirety. This was performed on a Cary-instrument using the appropriate corrections. Steady-state fluorescence and intrinsic anisotropy measurements were performed on a Fluorolog-3 from Horiba Jobin Jvon. For steady-state fluorescence measurements, solutions of 100 nM APTS and APTS-MT were made to avoid inner filter effects in TRIS buffer. Excitation and emission scans were performed to determine the fluorescence spectra of APTS-MT.

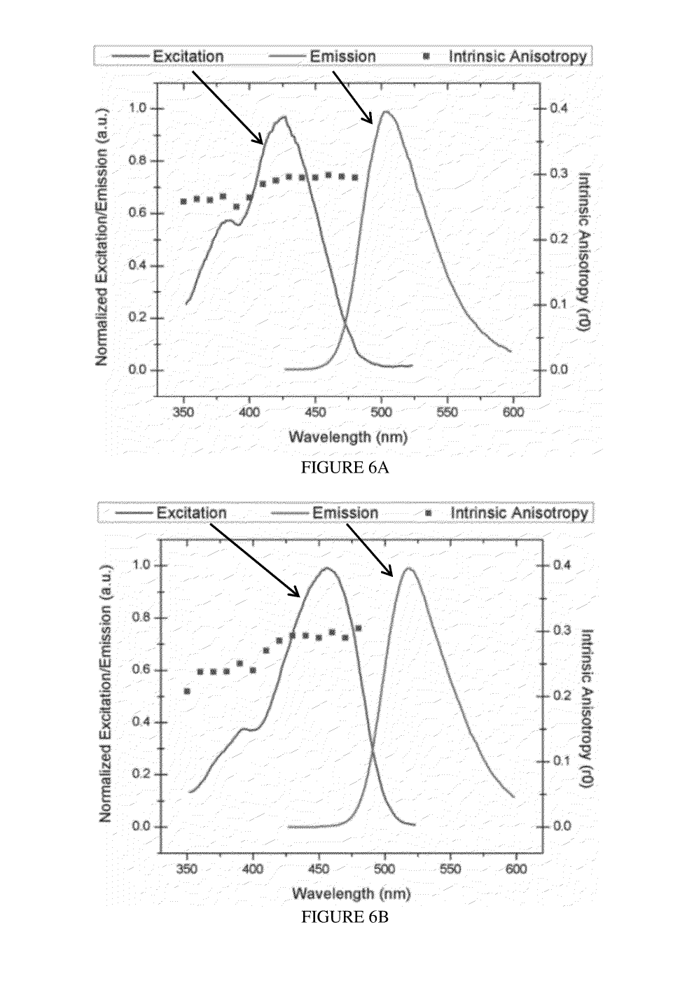

Intrinsic anisotropy measurements were performed by adding the fluorescent molecule of interest in a 50% glycerol solution at a concentration of 100 nM and setting the temperature to 5° C. This effectively slowed the rotation of our fluorescent molecules to negligible amounts (while in the excited state) which can allow the steady-state anisotropy value to be equivalent to the intrinsic anisotropy. Quartz cuvettes were used to avoid birefringence effects on the measured anisotropy. The anisotropy was recorded by collecting the emitted fluorescence at 520 nm with 5 nm bandpass. The G-factor was calculated for each sample. Ten measurements were taken for each sample, and the recorded anisotropy was the average of those measurements. FIG. 6A and FIG. 6B show the results of this steady-state fluorescence characterization for APTS (FIG. 6A) and APTS-MT (FIG. 6B).

As illustrated, the fluorescence of unconjugated APTS shows an excitation maximum at ˜425 nm and an emission maximum at ˜505 nm. After conjugation, the APTS-MT rationally designed fluorescent ligand shows an excitation maximum at ˜460 nm and an emission maximum at ˜520 nm. APTS-glucose shows similar spectra to APTS-MT. All spectra are corrected for the instrument response. This shift in the fluorescence spectra upon conjugation is due to the reorientation of the electron density of the pyrene backbone, and the shift in absorbance has commonly been used to identify via unique populations during chromatography. The fluorophore shows a relatively high stokes shift of approximately 70 nm, which allows for a large band of fluorescence wavelengths to be collected to improve without problems arising from scattered light.

Regarding the steady-state anisotropy, the fluorophores are effectively immobilized for the given fluorescence lifetime under these conditions. The intrinsic anisotropy is relatively stable for both conjugates near the excitation maximum, with a value of ˜0.3 ns. If a longer-lifetime fluorophore was used, higher viscosities and lower temperatures would need to be used.

Fluorescence Characterization of APTS-MT—Fluorescence Lifetime and Dynamic Anisotropy

The fluorescence lifetime and dynamic anisotropy data were collected using a DeltaFlex time-correlated single photon counting system from Horiba that was equipped with a 482 nm DeltaDiode pulsed source. The emission was set to 520 nm with the slit-width allowing 8 nm bandpass. A 500 nm high-pass filter was used to avoid any scattered excitation light. The effect that ConA binding has on the rotational correlation lifetime of APTS-MT was studied using a solution of 200 nM APTS-MT with and without 1 μM ConA present. Quartz cuvettes were used to avoid birefringence effects on the anisotropy measurements. Neutral density filters were used to adjust the fluorescence intensity to allowable levels. For fluorescence lifetime measurements, the excitation polarizer was in the vertical orientation and the emission polarizer was set to the magic angle (˜54.7°). Data was taken until the maximum counts were 10,000. Silica particles in DI were used as the sample to scatter light to determine the pulse-shape of the 482 nm source. For dynamic fluorescence anisotropy measurements, the fluorescence emission was collected in parallel and perpendicular configurations until the difference of the first point was 15000 counts. The fluorescence intensity decays were analyzed with the Decay Analysis Software (v. 6.6) from Horiba, and fits were used according to the expected distribution in the solution. The dynamic anisotropy decays were analyzed by using a reconvolution algorithm to determine the best-fit rotational correlation lifetimes. The fluorescence lifetime decay analysis indicated a single exponential decay of ˜5.3 ns.

The dynamic anisotropy and rotational correlation lifetimes of APTS-MT with and without ConA were also assessed. The reconvolution algorithm for the dynamic anisotropy displays rotational correlation lifetimes for the free and bound APTS-MT at ˜1 ns and ˜20 ns. The relative ratio between r1 and r2 is the relative fluorescence intensities between the two populations when the sample is initially pulsed. Therefore, if you assume that the quantum yield does not change upon binding and the solution is at steady-state binding, this is a measure of the relative concentrations of the APTS-MT in solution. These values suggest that at 1 μM, approximately 50% of the APTS-MT is bound to ConA, which is approximately what is expected with regard to the affinity.

These rotational correlation lifetimes are a measure of how fast the fluorescent particle is rotating in free solution. It would be ideal, therefore, for the fluorescence lifetime to be between the rotational correlation lifetime of the bound fluorescent ligand and the free fluorescent ligand to maximize the change of in the steady-state anisotropy.

Binding Characterization

A filter-based fluorescence microplate reader that was equipped with polarizers and the appropriate fluorescence filters for APTS was used to perform binding studies. The equilibrium-binding between ConA and APTS-MT was performed by loading a microplate with serial dilutions of ConA and the same concentration of APTS-MT (500 nM). The plate was allowed to reach equilibrium at room temperature (22° C.), and scans were performed in the perpendicular and parallel directions. Background was subtracted from each value. The G-factor was calculated from the fluorescence anisotropy for free APTS-trimannose at a value of 0.03, and this G-factor was used for the remaining experiments. Assuming the change in fluorescence lifetime upon binding to ConA to be negligible, the ConA-dependent anisotropy was calculated. These results were fit with a Boltzmann curve to determine the association constant to be 5.61*10⁶ M⁻¹.

Implementation of Rationally-Designed Fluorescent Ligands—Comparison to Ideal Characteristics

The experimentally determined characteristics of the APTS-MT were plotted on 2D sensitivity maps to predict its expected ability to optimize the affinity and transduction sensitivity mechanisms (not shown). The analysis demonstrated that APTS-MT is expected to have significantly improved results when compared to the results from the 4 kDa FITC-dextran.

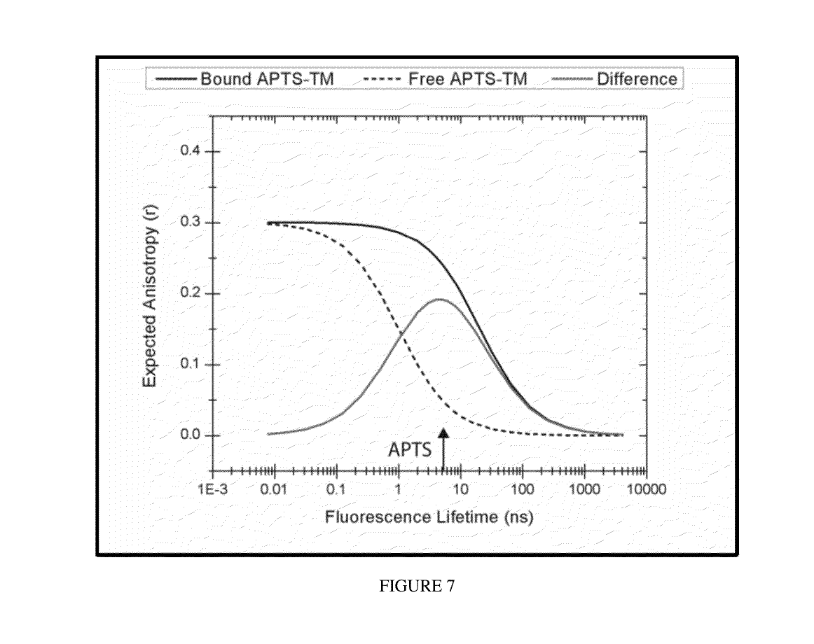

The suitability of the fluorescence lifetime to transduce the binding event can also be shown by directly comparing it with a curve from the measured rotational correlation lifetimes. The steady-state anisotropy for each rotational correlation lifetime was predicted for the full range of possible fluorescence lifetimes. FIG. 7 illustrates the difference between these two curves (see bell-shaped curve). The ideal fluorophore for a specific change in rotational correlation lifetimes would display a fluorescence lifetime that generates the largest change in the steady-state anisotropies. The analysis indicated that the APTS-MT fluorescence lifetime of 5.3 ns matches the peak of that difference curve (not shown), making it ideal to optimize the transduction mechanism of the fluorescence assay.

Implementation of Rationally-Designed Fluorescent Ligands—Fluorescence Anisotropy Based Assay

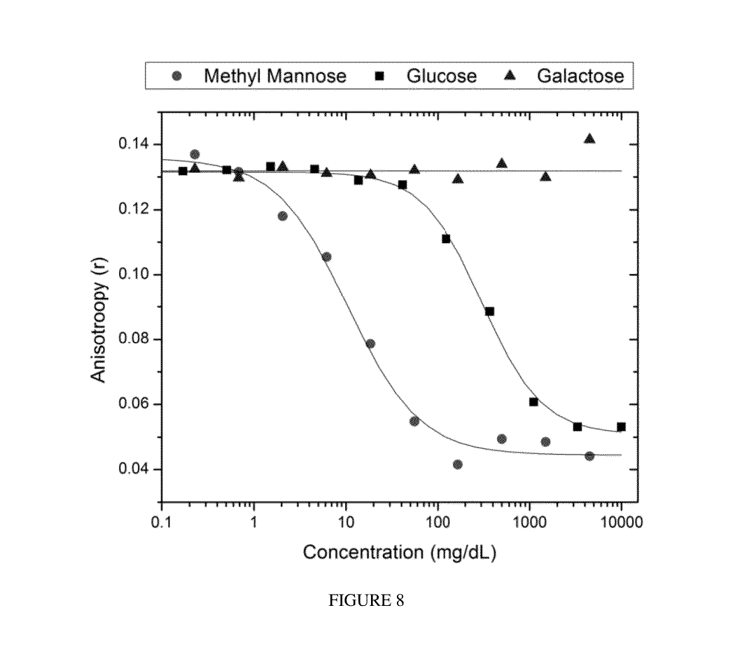

Sugars: Using the results from the modeled assay, the concentrations of the binding assay were chosen to be 200 nM APTS-MT and 1 μM unlabeled ConA. Following a similar strategy as the affinity-binding studies between APTS-MT and ConA, microplate wells were loaded with this assay with varying concentrations of methyl mannose, glucose, and galactose from ˜0.2 mg/dL to ˜10,000 mg/dL. The assay was given an appropriate time to reach equilibrium at room temperature (22° C.) and the steady-state anisotropy was scanned using the filter-based fluorescence microplate reader. The results are displayed in a semi-log plot in FIG. 8.

The responses in FIG. 8 show that the binding of the APTS-MT to ConA is effectively inhibited by monosaccharides that are known to only bind to the primary binding of ConA (mannose and glucose). In addition, methyl mannose is known to have a binding affinity that is ˜20-40 times higher than glucose. The concentration that causes a 50% reversal of the binding is on that order of increase for glucose. This is what is expected from the Chung-Prusoff equation for competitive binding. Lastly, galactose has no response on the assay, which is also to be expected. Galactose shows no affinity to ConA, therefore it is not expected to displace the APTS-MT. This set of results is highly encouraging and suggests that the APTS-MT is truly undergoing true competitive binding.

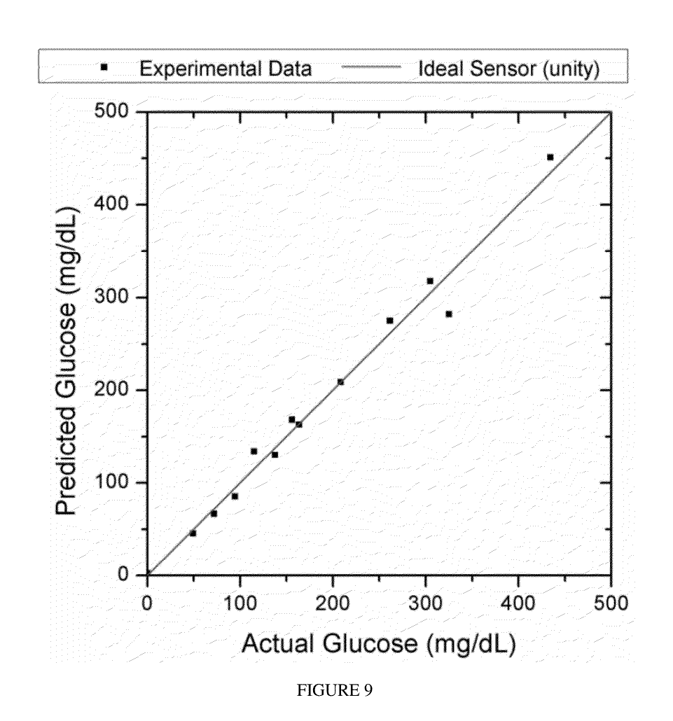

Calibration and Prediction of Glucose: Additional anisotropy experiments were performed with the assay to have more measurements in the physiologically relevant glucose concentrations. The actual glucose concentrations were determined on a YSI biochemistry analyzer. These anisotropy values were used to generate a calibration fit via the typical competitive binding equation, and this equation was used to predict the glucose concentrations. The predicted glucose vs. actual glucose (using the same data set) curve is shown in FIG. 9. This shows a standard error of calibration of 8.5 mg/dL and a mean absolute relative difference (MARD) of 6.5% across physiologically relevant glucose concentrations. In comparison to the prediction vs. actual plot of the assay based on the 4 kDa FITC-dextran, the points using the APTS-MT are much closer to the central line. The slight fluctuations are expected to be a pipetting error rather than an error with the assay.

### SUMMARY

The data described in this Example demonstrate that the rationally designed trimannose ligand with a single fluorescent moiety, as used in an anisotropy-based assay, overcomes the issues described for the existing ConA-based glucose sensing systems.

### Example 3

### Rationally Designed Fluorescent Ligand for ConA-Based FRET Assays

### Introduction

The generation of a rationally designed fluorescent ligand is described above, wherein the ligand is incorporated into an anisotropy-based assay for glucose sensing. The present Example describes the successful incorporation of the rationally designed fluorescent ligand into a FRET-based assay for glucose sensing.

### Results/Methods/Discussion

Even though this optimized anisotropy-based assay transduces the glucose concentrations effectively, an assay based on FRET has the potential to increase the sensitivity further. The anisotropy-assay is only as good as the polarizers that are being used. For example, in the above description, polarizers with an extinction of 6000:1 were used. Even with improved polarizers, there will be considerable more error in an optimized anisotropy assay than in an optimized FRET assay. As a result, a FRET assay is a desirable platform in which the rationally designed fluorescent ligand can be used as the donor.

The APTS-MT is expected to be extremely well-suited to translate to a FRET-based assay because it is the first fluorescent ligand for ConA-based assays that displays a fluorophore directly next to the only moiety to which ConA can bind. Traditional ligands paired with ConA (such as 70 kDa dextran) have been 10 nm in diameter and have displayed multiple places for ConA to bind. Upon binding of an acceptor-labeled ConA to the multivalent fluorescent ligand, the fluorophore on the fluorescent ligand could be various distances to the acceptor fluorophores on ConA. Because of the possible distances between donor and acceptor fluorophores, this can result in a binding event that appears to still be in free solution.

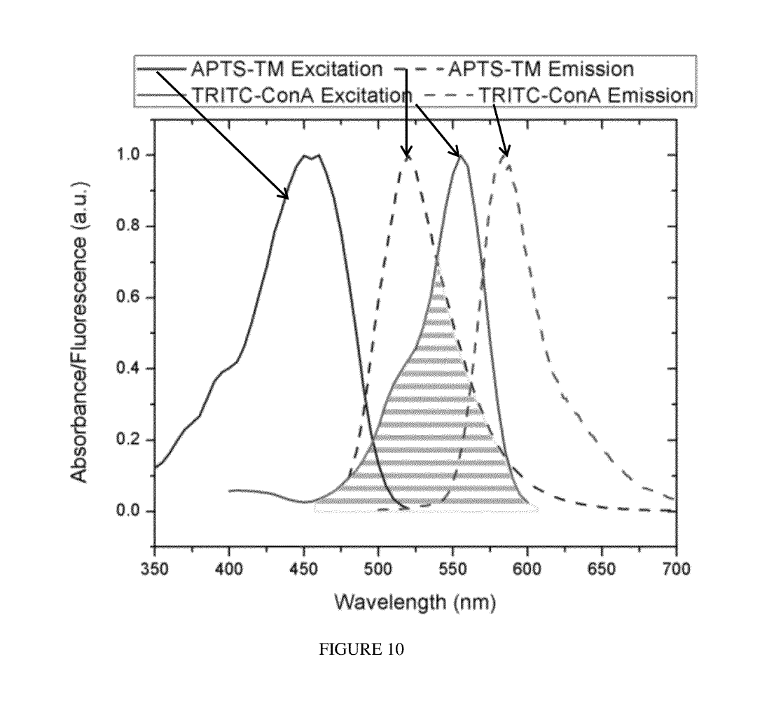

This APTS-MT avoids that fate by bringing the fluorophore to within ˜1 nm of ConA's binding site every time that ConA binds the APTS-MT. Therefore, with the appropriate acceptor fluorophore on ConA and with high enough degree of labeling, the expected FRET efficiency upon binding can be very high. For these experiments, TRITC was used as the acceptor fluorophore and was labeled to ConA. The degree of labeling was approximately 4 fluorophores per ConA. Fluorescence measurements were performed on an ISS PC1 spectrofluorometer. The concentrations used to obtain the excitation/emission spectra for APTS-MT and TRITC-ConA was 100 nM and 1 μM, respectively. This is expected to have a Förster radius similar to FITC/TRITC (˜5 nm). The excitation and emission properties, and their overlap, are shown in FIG. 10.

A 100 nM APTS-MT solution was then made in TRIS buffer for titration experiments. Increasing TRITC-ConA concentrations were added to the cuvette, and fluorescence measurements were taken (not shown). The resulting spectra display a decrease in the donor fluorescence (APTS-MT) and an increase in the acceptor fluorescence (TRITC-ConA).

The fluorescence intensity at 520 nm (from the APTS-MT) for each TRITC-ConA concentration was normalized to the fluorescence intensity at 520 nm in the absence of TRITC-ConA. These values were then plotted as a function of the APTS-ConA concentration (not shown). This data was fitted with Equation 3 to account for both of the components that decrease the fluorescence intensity. This includes: (1) the energy transfer associated with binding to the TRITC-ConA, and (2) the effects of adding the acceptor fluorophore to the bulk solution. This fitting shows that the affinity of the APTS-MT and TRITC-ConA binding is 3,522,367 M⁻¹, which is slightly lower than the affinity obtained from anisotropy measurements with unlabeled ConA. The fitting also showed that b was equal to 0.1968, which indicates the fraction of the initial intensity that is expected to be seen if 100% of the APTS-MT was bound. This value can be used as a measure of the average FRET efficiency of the bound population, and suggests that the efficiency for this competitive binding pair is ˜80.3%. This confirms that the rationally designed fluorescent ligand can effectively bring the fluorophore to within the Förster radius of the FRET pair upon binding.

y=((a−b)/(1+(x/c))+b)+d*x (3)

The acceptor-peak fluorescence intensity should show a similar affinity; however, the tail of the emission from the APTS-MT overlaps with the emission of the TRITC-ConA. Therefore, spectral un-mixing was performed to study the acceptor fluorescence (not shown). The peak of this emission at 585 nm of each of these curves was plotted as a function of the TRITC-ConA concentration (not shown). Again, this is fit with Equation 3 to account for both of the components that increase (in this case) the fluorescence intensity. This fit shows that the affinity of the APTS-MT and TRITC-ConA binding is 3,258,390 M⁻¹, which agrees well with the information obtained from the donor peak.

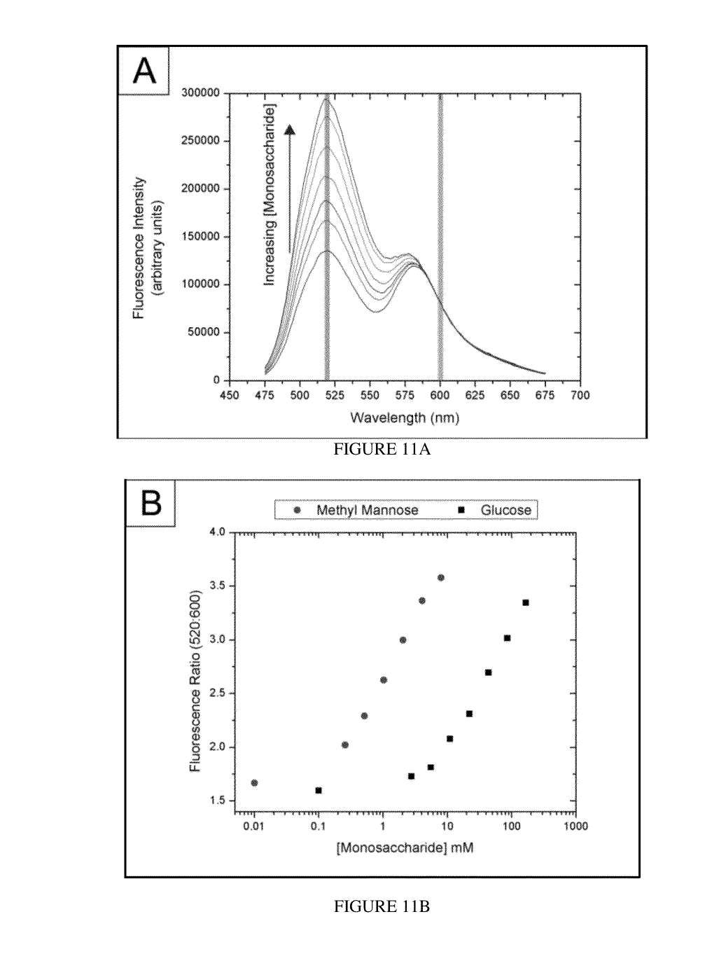

A FRET-based competitive assay was then generated that was comprised of 100 nM APTS-MT and 1 μM TRITC-ConA in TRIS buffer. This was used to track the competitive binding to various sugars with the fluorescence. Upon titration of highly concentrated aliquots of methyl-alpha-mannose and glucose, sufficient time was given to allow for equilibrium to be reached before fluorescence measurements were made. Excitation was performed at 450 nm with a 15 nm bandpass and emission was collected from 475 nm to 675 nm to collect both APTS and TRITC's emission. The responses are shown in FIG. 11A. The blue (left vertical line) and red (right vertical line) lines indicate the wavelengths (520 nm and 600 nm) that are used to generate the fluorescence ratio (520:600).

The resulting spectra show that increasing concentrations of the monosaccharides increase the relative fluorescence of the donor-fluorophore, which is characteristic of competitive binding. The acceptor emission appears to increase slightly with increasing concentrations of monosaccharides, but this is due to the spectral overlap of the tail of the emission of APTS-MT at longer wavelengths. By taking a ratio of the main peak of the donor fluorescence (at 520 nm, left vertical line) and the tail of the acceptor fluorescence (at 600 nm right vertical line), a ratiometric signal was generated. These ratiometric responses show a similar effect to what is seen for the anisotropy-based assay as seen in the semilog plot in FIG. 11B. The assay responds to lower concentrations of methyl-mannose than glucose, which is to be expected because of ConA's higher affinity to methyl-mannose.

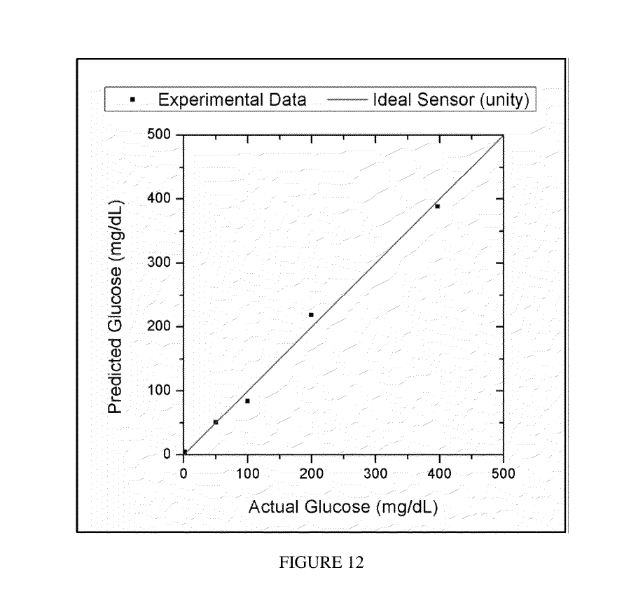

The glucose dependent fluorescence ratio was generated on a linear plot with the best fit data using the aforementioned curve for competitive binding data (not shown). The r2 of this fit is 0.9986. This specific assay shows a linear response across the physiological glucose concentrations. The predicted glucose vs. actual glucose (using the same data set) curve is shown in FIG. 12. Again, the error associated with this fit is most likely due to pipetting error. This assay could be tweaked to maximize the response, as the effective IC50 for the fit is 792 mg/dL. The response could also be increased by labeling ConA with additional acceptor fluorophores to increase the FRET efficiency upon binding to the APTS-MT.

### SUMMARY

Example 2 introduced a modular approach to the design of a rationally designed fluorescent ligand to achieve the desired qualities as outlined in the previous chapter. The core trimannose was identified as a potential high-affinity ligand for ConA due to its binding to the extended binding site of ConA (also referred to as a subsite). This core trimannose showed no aggregation when paired with ConA, and a fluorescent ligand was synthesized to present a single trimannose moiety. This rationally designed fluorescent ligand was characterized and shown to be in the optimized regions of the sensitivity maps as previously defined. The assay was implemented into anisotropy-based assays for glucose. In this example, the rationally designed ligands were successfully implemented into FRET-based assays for glucose. These assays showed a dynamic response across physiological glucose concentrations, where the error was most likely due to pipetting rather than instrument error.

The described ligands are expected to remain stable when paired with ConA because they show a single sugar motif, to avoid crosslinking between multivalent components. Furthermore, the ligands are negatively charged to avoid electrostatic effects. Finally, the fluorophore is near the binding motif of the ligands to allow for maximization of the FRET-efficiency upon binding.

### Example 4

### Second Generation of Rationally Designed Fluorescent Ligand for ConA-Based FRET Assays

### Introduction

The previous Examples described the development of a rationally designed fluorescent ligand concept to overcome the irreversibility/aggregation problems that have plagued ConA-based glucose sensing assays. As described, this rationally designed fluorescent ligand is advantageous because it: (1) displays a single moiety that can bind to ConA's full binding site, (2) is negatively charged, and (3) is fluorescently labeled. However, an obstacle remains for this strategy to be used in a continuous glucose sensor: the size of this rationally designed fluorescent ligand must be increased to prevent leaching from the semi-permeable membrane that allows for glucose diffusion when in situ.

This Example describes the development of a second generation of rationally designed ligand for a ConA-based glucose sensing assay. The second generation ligand incorporates a large protein scaffold, in this case ovalbumin, on which ConA-binding component and a reporting component can be attached and to provide for increased size. Ovalbumin was identified as a viable template/scaffold for a 2nd generation rationally designed fluorescent ligand because: (1) it has a single glycosylation site (a single asparagine residue at position 292 (Asp-292)) that can present a high-mannose glycan (e.g., which contains the core trimannose), (2) it is negatively charged at physiological pH (with an isoelectric point of 4.5), (3) it has a molecular weight of 45 kD, and (4) it has numerous lysine residues that can be labeled with an amine-reactive fluorophore, in addition or in lieu of using the N-terminal glycine (Gly-1). This strategy can be used as a cost-effective method to generate the bulked-up, 2nd-generation, rationally designed fluorescent ligand without a significant number of synthetic steps.

### Results/Methods/Discussion

Azadioxatriangulenium (ADOTA) Fluorophore

This preliminary study uses the Azadioxatriangulenium (ADOTA+) fluorophore as ADOTA+ has fluorescence properties ideal for tracking the binding between ovalbumin and ConA. See Laursen, B. W. and Krebs, F. C., Angewandte Chemie—International Edition 39:3432-3434 (2000); Laursen, B. W. and Krebs, F. C. Chemistry—A European Journal 7:1773-1783 (2001); and Dileesh, S. and Gopidas, K. R., Journal of Photochemistry and Photobiology A: Chemistry 162:115-120 (2004), each of which are incorporated herein by reference in their entireties. See also U.S. Patent Application Publication No. 2006/0211792, incorporated herein by reference in its entirety, for disclosure relating to fluorophores. ADOTA+ displays excitation and emission maxima at 540 nm/560 nm with a fluorescence lifetime of ˜23 ns. It has moderate brightness with a molar extinction coefficient of 9,800 cm⁻¹M⁻¹ and a quantum yield of 0.49 (determined in acetonitrile), with an intrinsic anisotropy has been shown to be 0.38. See Thyrhaug, E., et al., Journal of Physical Chemistry A 117:2160-2168 (2013), incorporated herein by reference in its entirety. The structure of ADOTA-NHS is shown below:

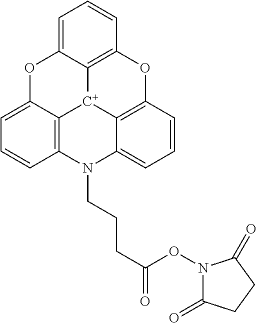 [SMILES: O=C(CCCn1c2cccc3oc4cccc5oc6cccc1c6[c+](c45)c32)ON1C(=O)CCC1=O]

With these fluorescence properties, ADOTA+ can allow binding studies between relatively large molecules to be tracked with anisotropy, and it can be used to avoid the shorter fluorescence lifetimes that are common for endogenous fluorophores.

Preparation of ADOTA-Labeled Glycated Ovalbumin

The ConA-based assay, as described herein, tracks the fluorescence intensity from the fluorescently labeled ovalbumin. As a result, it is essential that all of the ovalbumin can bind to ConA. If a portion of the fluorescently labeled ovalbumin cannot bind to ConA, it will generate background signal that is not sensitive to glucose concentration and will increase the error of the sensor. The glycans on proteins can display a significant amount of variation, and a large fraction of proteins that can be glycated do not actually display glycans.

The glycated fraction was separated from the non-glycated fraction of the ovalbumin sample by performing affinity-chromatography with ConA-functionalized resin. The protein elution from the column was tracked by monitoring the absorbance at 280 nm. After the non-glycated fraction eluted from the column, high concentrations of mannose were used to elute the glycated fraction. This glycated fraction was collected and dialyzed in sodium bicarbonate buffer to prepare it for labeling.

The glycated fraction of the ovalbumin was labeled with the ADOTA-NHS according to traditional amine-reactive protocol. Briefly, ADOTA-NHS was dissolved in DMSO, added dropwise to the solution of glycated ovalbumin in sodium bicarbonate buffer (pH 9 to deprotonate the primary amines), mixed well, and allowed to react for 1 hour in the dark at room temperature. Afterwards, the free dye was removed via dialysis against TRIS buffer, and the solution was filtered with syringe filters. In other samples, the free dye was removed via size exclusion chromatography (SEC) with the HPLC pump system. In such approaches, if ADOTA-OVA aggregates with itself, potential methods to decrease non-specific self-interactions can be employed to minimize or prevent inefficiencies of the resulting ligand.

To determine the concentrations and degree of fluorescent labeling, UV/VIS absorbance measurements were performed in TRIS buffer. Using the molar extinction coefficients, the concentration and degree of labeling of the ADOTA-OVA was determined. Similar UV/VIS studies were performed with the AF647-ConA that was purchased from Invitrogen. The degree of labeling of the ADOTA-OVA was determined to be approximately 1.5 ADOTA fluorophores per glycated ovalbumin. The degree of labeling of the AF647-ConA was determined to be approximately 3 AF647 fluorophores per ConA.

An expected 3D representation of the ADOTA-glycated ovalbumin was developed by using the x-ray crystallography structure of ovalbumin found published (PDB: 3VVJ) and altering it in the Maestro software. A high mannose glycan was attached to the asparagine residue (Asp-292). Two ADOTA fluorophores were attached to two of the primary amines coming from any of the lysine groups. See the representative amino acid sequence of ovalbumin, set forth in SEQ ID NO:2.

Fluorescence Anisotropy with ADOTA-OVA

The 2D sensitivity map for the anisotropy transduction mechanism was previously generated as described above (not shown). Fluorescence anisotropy is typically limited to tracking small fluorescent ligands to large receptors. Because ovalbumin is relatively large in size (˜45 kDa), most organic fluorophores would not show a change in anisotropy upon binding as they typically display a fluorescence lifetime of a few ns. However, ADOTA can allow the equilibrium binding of ovalbumin and ConA to be tracked with fluorescence anisotropy because of its longer fluorescence lifetime (˜20 ns). Accordingly, despite the size of ovalbumin, it is predicted on the 2D sensitivity map to provide a reasonable anisotropy sensitivity when using ADOTA as the fluorophore (not shown).

Being able to track the ovalbumin-ConA interactions with anisotropy allows the association constant to be determined in a realistic environment. This association constant can then be confidently used in the competitive binding model to generate an optimized assay for the desired glucose concentration range. The fluorescence anisotropy of a 500 nM solution of ADOTA-glycated ovalbumin was tracked as a function of ConA concentration using a Fluorolog 3 spectrofluorometer. This data was fit with a Boltzmann curve, and the affinity was determined to be 587,000 M⁻¹. This anisotropy could be used as a glucose-sensor, as described in the previous Examples; however, the limited change in anisotropy for this competitive binding pair, due to the characteristics of the Ovalbumin scaffold, may provide suboptimal glucose prediction.

FRET-Based Studies with ADOTA-OVA

One eventual goal for the ADOTA-OVA is to be paired with ConA behind a semi-permeable membrane to create a fluorescent sensor that can track glucose concentrations in vivo. To track the competitive binding with energy transfer, an acceptor fluorophore was added to ConA to allow the binding of the ligand to ConA to be detectable via FRET-based signal. For this work, AF647 was chosen as a suitable dye to allow for spectral overlap while minimizing the direct excitation of the acceptor fluorophore.

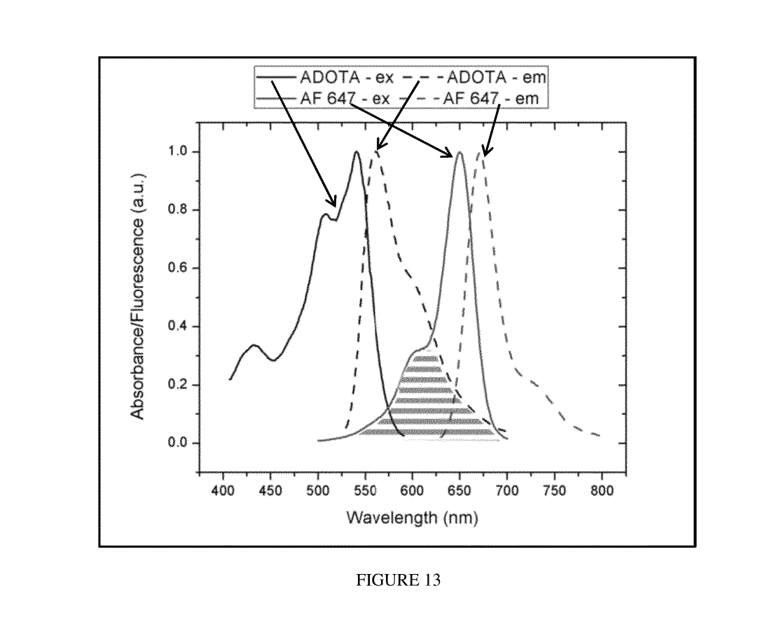

The 3D representation of the interaction between the ADOTA-glycated ovalbumin and AF647-ConA was generated with the Maestro software (not shown). This assists ascertaining the distances between the donor and acceptor fluorophores upon binding of a portion of the high-mannose glycan to the full binding site of ConA. The steady-state excitation (solid) and emission (dashed) spectra for ADOTA (left two peaks) and AF647 (right two peaks), and their overlap as required for energy transfer in FRET signaling, are shown in FIG. 13.

Stopped Flow Measurements

A stopped-flow technique is one that mixes two solutions together very quickly to allow for the kinetics of a solution to be monitored over time. This can be useful to study receptor-ligand interactions of a fluorescence-based assay that is engineered to change its fluorescence properties upon binding. For such an assay, the receptor is loaded into one syringe and the ligand is loaded into the other syringe. The solutions are driven to the mixing chamber which occurs in a cuvette. If this cuvette is placed in a typical spectrofluorometer, the fluorescence intensity can be tracked as a function of time. It is also possible to monitor anisotropy as a function of time, but this measurement requires the spectrofluorometer to be in a T-format configuration with two emission pathways where one collects perpendicular fluorescence and the other collects parallel.

Using the FRET-based assay with ADOTA-OVA and AF647-ConA, the intensity of the ADOTA-OVA is expected to decrease upon binding to AF647-ConA. It is important to note that the concentration of interest is the final concentration. These studies used identical syringes for the receptor and ligand. So, the concentrations in the cuvette were half of what was loaded into the syringes. Originally, the ADOTA-OVA was loaded at concentration of 1 μM. Three different ConA concentrations were loaded into the receptor chamber (0.33 μM ConA, 0.66 μM ConA, and 2 μM ConA). The trigger from the stopped-flow accessory was an input to the software to begin fluorescence collection. The excitation was set to 500 nm, and the emission was set to 550 nm to measure the emission peak from the ADOTA-OVA. The fluorescence intensity was measured every 0.1 second for 100 seconds.

These time-dependent fluorescence decays can then be normalized to the initial fluorescence intensity. The initial intensity decreases due to two primary reasons, neither of which is due to binding events. Because the AF647 can be directly excited by the excitation light, the increasing concentrations of the AF647 cause a decrease in the excitation light that actually gets to the ADOTA fluorophore. In addition, because of the spectral overlap of the ADOTA emission and AF647 excitation, there exists the possibility of the ADOTA-emission being reabsorbed by AF647. While it is also possible that diffusion-dependent energy transfer could occur, the concentrations are not high enough to make this occur on the time-scale that ADOTA is in the excited state. After normalizing the fluorescence decays to the initial fluorescence, they can be fit with a single exponential decay.

The traditional practice for stopped flow measurements is to plot these apparent rate constants as a function of the receptor concentration (not shown). The best fit line to this data generates the expected kₒₙ and k_{off} rates. The kₒₙ rate is the slope of that line and the k_{off} rate is the y-intercept at low ovalbumin concentrations. These results show that the association constant (equivalent to kₒₙ/k_{off}) is approximately 554,000 M⁻¹, which is very similar to the previous results that were determined with anisotropy. By knowing the kinetic rate constants, the time-dependent competitive binding can be determined using numerical methods.

Steady-State FRET Based Glucose Assay

To test the steady-state glucose response, the assay was chosen to have 500 nM ADOTA-OVA and 1 μM AF647-ConA. This assay was loaded into a cuvette of TRIS buffer, and highly concentrated glucose aliquots were added to minimize dilution effects. Upon each addition, the solutions were mixed well and given time to equilibrate. At each glucose concentration (0 mg/dL-500 mg/dL)), the steady-state fluorescence was measured on a Fluorolog 3. Excitation was at 500 nm with a 10 nm bandwidth. Emission was collected from 530 nm to 750 nm with a 5 nm bandwidth.

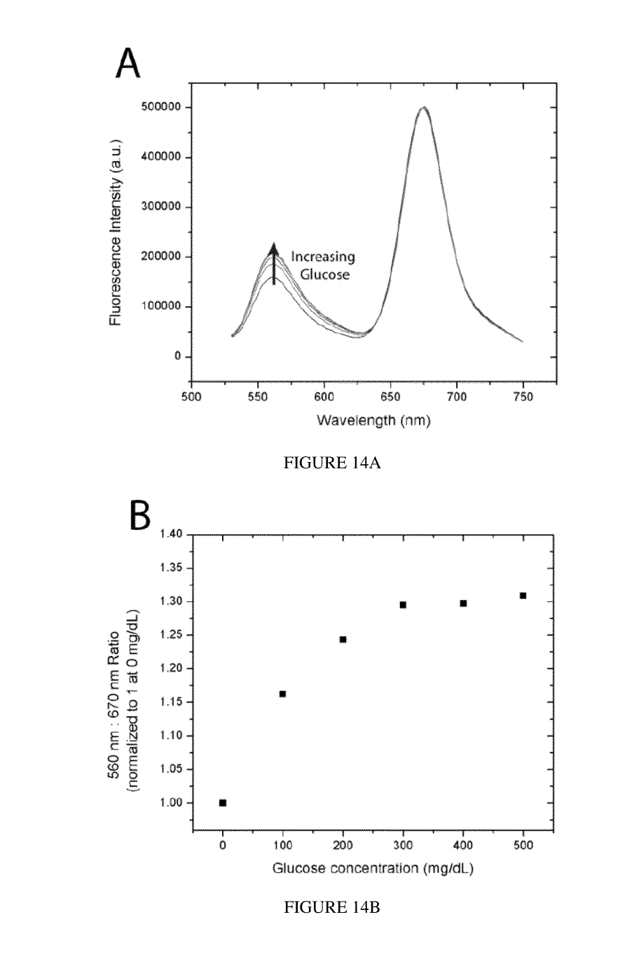

The assay displayed glucose-dependent fluorescence spectra that are characteristic of competitive binding (see FIG. 14A and FIG. 14B). The fluorescence of the ADOTA-OVA increased with increasing concentration, indicating that the concentration of ADOTA-OVA undergoing FRET (bound population) was decreasing. The emission of each fluorophore is sufficiently separated to minimize the effect that the changing fluorescence intensity of ADOTA has on the AF647 fluorescence. The ratio of the donor to acceptor maxima (560 nm to 670 nm) as a function of glucose concentration is also shown. This shows that the fluorescence of this ADOTA-OVA and AF647 ConA assay primarily changes from 0-300 mg/dL, and then begins to flatten out.

Despite the aggregate size of the second generation ligand, this assay still shows a significant increase in efficient FRET signaling across physiological glucose concentrations (˜30% fluorescence increase), and it could potentially be increased further by increasing the AF647-ConA concentrations further. This could also be increased by site-specific labeling of the N-terminus of the ovalbumin. In the crystal structure, the N-terminus appears to be fairly close to where the high-mannose glycan resides and could allow for improved FRET efficiency. Reference to the 2D sensitivity map previously generated (not shown), indicates the sensitivity of signaling and the ability to increase sensitivity. Specifically, the analysis indicates that increasing the concentration of the AF647-ConA, this fluorescence response is expected to increase further.

FRET-Based Glucose Assay with Fluorescence Lifetimes

An assay was generated using 500 nM ADOTA-OVA and 4 μM AF647-ConA for FRET-based studies where the fluorescence lifetime decays were analyzed instead of the steady-state intensity. The fluorescence lifetime was collected using a DeltaFlex time-correlated single photon counting system from Horiba that was equipped with a 482 nm DeltaDiode pulsed source. The emission was set to 540 nm with the slit-width allowing 8 nm bandpass. A high-pass filter of 500 nm was used to block scattered excitation light. The excitation polarizer was in the vertical orientation and the emission polarizer was set to the magic angle. Data was taken until the maximum counts were 1,000. Silica particles in DI were used as the sample to scatter light to determine the pulse-shape of the 482 nm source. The fluorescence decays of the 500 nM ADOTA-OVA and the assay of the 500 nM ADOTA-OVA with 4 μM AF647-ConA (not shown).

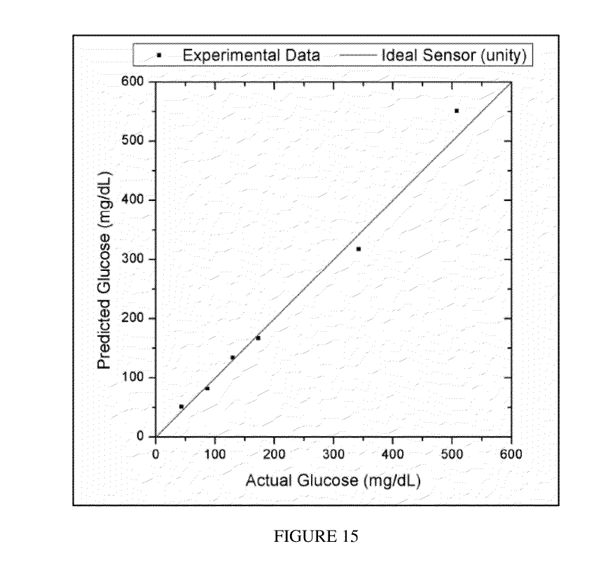

The time-dependent fluorescence decays were fit with three exponentials. Two of these lifetimes were fixed: one for the shorter lifetime at 2.07 ns and the other for the longer lifetime at 18.19 ns. A third lifetime (on the order of picoseconds) was also implemented in the fit to account for the scatter that was seen in the experiments. The full decay of each solution was fit using with this equation by minimizing the residuals. The weighted intensities at time zero of the short and long lifetime decays (B1 and B2) were then recorded as a function of glucose concentration. These values are expected to be a function of the equilibrium binding between ADOTA-OVA and AF647-ConA. The population that is capable of undergoing FRET is expected to primarily display the shorter lifetime. The population that does not undergo FRET is expected to primarily display the longer lifetime. Therefore, B2/(B1+B2) should correlate to how much of the ADOTA-OVA is free. The competitive binding equation was again used to fit this data (not shown), and a best fit was generated with an IC50 of 386.9 mg/dL. The predicted glucose vs. actual glucose (using the same data set) curve is shown in FIG. 15. The standard error of calibration was 8.25 mg/dL, and the calibration MARD was 4.19%. Improvements could potentially be made on the assay to improve the average FRET efficiency of the bound ovalbumin population, for instance by increasing the degree of labeling on ConA or performing site-specific labeling on the ovalbumin with the ADOTA+ fluorophore.

### SUMMARY

This Example describes the incorporation of the naturally occurring ovalbumin as the core scaffold component of a 2nd generation rationally-designed fluorescent ligand to be paired with ConA in a fluorescence glucose sensor. The glycated fraction of ovalbumin was separated with affinity chromatography and labeled with the long-fluorescence-lifetime ADOTA+ dye to allow the binding to be tracked via fluorescence. A FRET assay was generated by pairing ADOTA-OVA with AF647-ConA that demonstrated a robust fluorescence response across physiologically relevant glucose concentrations. This response could be tracked by looking at the changes in fluorescence lifetime of the ADOTA fluorophore and the steady-state fluorescence intensity. Thus, this Example demonstrates that the ADOTA-OVA fluorescent ligand can allow the translation of the rationally designed fluorescent ligand concept into a continuous glucose monitoring device.

### CONCLUSION

In summary of the above example, the goal of this research was to engineer a homogeneous ConA-based competitive binding assay that could remain stable in free solution and/or in situ and accurately predict the glucose concentration of that environment over time from the fluorescence signal of the assay.

Among existing technologies are assays that use glycosylated dendrimers as the competing ligand. Different versions of the glycosylated dendrimer were tested in an attempt to identify a specific version that enabled the assay to most effectively track physiological glucose concentrations. The assays that used this approach display problems with stability and reversibility in free solution and it showed several inconsistencies that have been attributed herein to electrostatic interactions and aggregation. Thus, alternative assay configurations were sought.

As described in Example 1, mathematical modeling was performed to identify the characteristics of an assay that could allow for dynamic fluorescence changes to physiological glucose concentrations. This work separated the recognition and transduction mechanisms of a fluorescent competitive binding sensor, and each mechanism was modeled independently. The transduction mechanism was chosen to be fluorescence anisotropy to make the system simpler and allow it to be linear. The sensitivity of the measured anisotropy is then the convolution of the sensitivities of each mechanism, making it necessary for both to respond accordingly. For the transduction mechanism, the steady-state anisotropy was modeled for fluorescent competing ligands of different molecular weight and fluorescent lifetimes. The expected steady-state anisotropy was modeled for the free-state and the bound-state (to tetrameric ConA) for each ligand, assuming each version was a perfect sphere at the molecular weight of the complex. For the recognition mechanism, an exact solution to the competitive binding equation was used for a set of affinities and concentrations. From these models, 2D sensitivity plots were generated for each mechanism to identify optimal assay configurations, and a combined model was made to predict the change in anisotropy for various assays.

The combined model was then validated by testing the glucose response of assays that paired 4 kDa FITC-dextran with different concentrations of ConA. After predicting the correct trends, this model was used to explain the previous problems associated with ConA-based glucose sensing assays in free solution. From this explanation, the hypothesis was postulated that the full potential of ConA-based assays can be shown if a fluorescent competing ligand is employed that achieves the high-affinity required without allowing for aggregation to occur with ConA. In addition, ideal characteristics were identified to maximize both the recognition and transduction mechanism.

As described in Example 2, the apparent dilemma between sensitivity and aggregation was overcome by identifying an alternative method to achieve the increased affinities. Instead of using proximity effects that requires a multivalent presentation of monosacccharides, the use of ConA's extended binding site was employed. The core trimannose is shown to form additional hydrogen bonds to the extended binding site while binding to the same primary binding site. A rationally designed fluorescent ligand was designed and synthesized to achieve the desirable characteristics as previously identified without allowing for aggregation to occur. A single presentation of the core trimannose showed no aggregation when paired with ConA, unlike dextrans and dendrimers.

A fluorophore (APTS) was attached to the reducing terminus of mannotetraose via reductive amination to generate a fluorescent ligand that presented a single core trimannose. This first generation rationally designed fluorescent ligand was fully tested to determine its fluorescent properties (intensity, intrinsic anisotropy, lifetime) as well as its binding affinity to ConA. Then, this rationally designed fluorescent ligand was paired with ConA in a fluorescence anisotropy based glucose sensor. As further described in Example 3, the rationally designed fluorescent ligand was paired with fluorescently labeled ConA in a FRET assay. The responses to several sugars were investigated, displaying that glucose and mannose concentrations can directly compete for binding sites on ConA with the rationally designed fluorescent ligand. This rationally designed fluorescent ligand (1) presented a single core trimannose moiety, (2) was negatively charged, and (3) held the fluorophore close to the trimannose moiety. This approach can allow the assay to remain stable over time, suggesting that this strategy could truly show the full potential of ConA-based assays. However, for this rationally designed ligand concept to be translated into a prototype device, the molecular weight must be increased to prevent leaching from the semi-permeable membrane.

Thus, as described in Example 4, in an attempt to generate a low-cost bulked-up ligand that can allow the rationally designed fluorescent ligand concept to be translated into a prototype device, various glycoproteins were explored. Ovalbumin, the primary protein found in egg-white, was been identified as a possible template/scaffold for a 2nd generation rationally designed fluorescent ligand because: (1) it has a single glycosylation site (see Asp-292 of SEQ ID NO:2) that can present a high-mannose glycan (e.g., which contains the core trimannose), (2) it is negatively charged at physiological pH (with an isoelectric point of 4.5), (3) it has a molecular weight of 45 kDa, and (4) it has numerous lysine residues that can be labeled with an amine-reactive fluorophore, in addition to or in lieu of using the N-terminal glycine (Gly-1). Affinity chromatography was used to collect only the glycated fraction, and this fraction was labeled with a red-emitting, long-lifetime fluorophore (ADOTA) to track the binding. Anisotropy studies were performed with unlabeled ConA and energy transfer studies paired the ADOTA-glycated ovalbumin with AF647-ConA. Many of these studies showed very promising glucose-sensing results.

An advantage of ADOTA-glycated ovalbumin as a ligand in a glucose sensing assay is that the ligand is amenable to further modification to enhance performance. For example, the ovalbumin can be stabilized using processes like PEGylation or by generating a purely synthetic ligand to further reduce, inhibit, or prevent aggregation, which is a demonstrated problem of the existing multivalent approaches. In addition, fluorophores could be chosen that excite/emit further into the red, to minimize the background from endogenous auto-fluorescence. Such resulting assays could be encapsulated within the desired matrix and has the potential to remain stable for long periods of time if it were paired with stabilized versions of ConA.

While illustrative embodiments have been described, it will be appreciated that various changes can be made therein without departing from the spirit and scope of the disclosure. Specific elements of any of the foregoing embodiments can be combined or substituted for elements in other embodiments. Furthermore, while advantages associated with certain embodiments of the disclosure have been described, other embodiments may also exhibit such advantages, and not all embodiments need necessarily exhibit such advantages to fall within the scope of the disclosure.

The embodiments of the invention in which an exclusive property or privilege is claimed are defined as follows:

1. A ligand for Concanavalin A, comprising:
(a) a Concanavalin A binding component that binds to the primary glucose binding site and part or all of an extended binding site on Concanavalin A; and
(b) a transduction component, wherein the binding component is coupled to the transduction component, and wherein the transduction component generates a detectable signal upon binding of the ligand to Concanavalin A.

2. The ligand of claim 1, wherein the binding component comprises one or more mannose moieties.

3. The ligand of claim 1, wherein the binding component comprises a trimannose moiety.

4. The ligand of claim 3, wherein the trimannose moiety is 3,6-Di-O-(α-D-mannopyranosyl)-D-mannopyranose.

5. The ligand of claim 1, wherein the binding component comprises a bimannose moiety.

6. The ligand of claim 5, wherein the bimannose moiety is 6-O-α-D-mannopyranosyl-D-mannopyranose or 3-O-α-D-mannopyranosyl-D-mannopyranose.

7. The ligand of claim 1, wherein the ligand has a binding affinity for Concanavalin A from about 10,000 to about 10,000,000 L/mol.

8. The ligand of claim 1, wherein the transduction component is a fluorophore, a Raman reporter, or a nanoparticle, or is electrochemically active.

9. The ligand of claim 1, wherein the transduction component generates a detectable signal by a transduction mechanism selected from fluorescence intensity, fluorescent resonance energy transfer (FRET), fluorescence anisotropy, fluorescence lifetime, Raman spectroscopy, and metal enhanced plasmonics.

10. The ligand of claim 1, further comprising a tether point for immobilization of the ligand to a structure or surface.

11. The ligand of claim 1, further comprising a proteinaceous scaffold.

12. The ligand of claim 11, wherein the binding component or the transduction component is coupled to the proteinaceous scaffold, or both the binding component and the transduction component are independently coupled to the proteinaceous scaffold.

13. The ligand of claim 11, wherein the proteinaceous scaffold has a net negative charge.

14. The ligand of claim 11, wherein the proteinaceous scaffold is or comprises ovalbumin, RNase B, or any derivative thereof.

15. A method for monitoring glucose in a sample, comprising:
detecting the competitive binding of a ligand to Concanavalin A in the sample, wherein the ligand has an affinity toward the primary glucose binding site and at least a portion of an extended binding site of Concanavalin A, wherein the ligand competes with glucose for binding to the primary binding site of Concanavalin A, and wherein a detectable signal is provided by a transduction component upon binding of the ligand to Concanavalin A.

16. The method of claim 15, wherein the detecting step comprises contacting the sample with the Concanavalin A and the ligand.

17. The method of claim 15, wherein the equilibrium binding of the ligand to Concanavalin A is inversely related to the glucose concentration in the sample.

18. The method of claim 15, wherein the sample is an in vitro or in vivo biological sample.

19. The method of claim 18, wherein the biological sample is blood, blood plasma, blood serum, extracellular fluid, interstitial fluid, or aqueous humor fluid.

20. The method of claim 15, wherein the detecting is performed in a continuous glucose monitoring assay.

21. The method of claim 15, wherein the ligand comprises the transduction component.

22. The method of claim 15, wherein the Concanavalin A comprises the transduction component.

23. The method of claim 15, wherein the ligand is the ligand of claim 1.

24. The method of claim 23, wherein each of the ligand and the Concanavalin A comprises a transduction component.

25. The method of claim 24, wherein the transduction component of the ligand and the transduction component of the Concanavalin A are capable of mutually interacting as a FRET pair.

26. A glucose monitoring system, comprising:
(a) Concanavalin A;
(b) a ligand having an affinity toward the primary binding site and all or part of the extended binding site of Concanavalin A, wherein the ligand effectively competes with glucose for binding to Concanavalin A; and
(c) a transduction component to signal the state of assay binding.

27. The system of claim 26, wherein the ligand comprises the transduction component.

28. The system of claim 26, wherein the ligand is the ligand of claim 1.

29. The system of claim 26, wherein each of the ligand and the Concanavalin A comprises a transduction component.

30. The system of claim 26, wherein the system is adapted to an implanted biosensor.

31. The system of claim 26, wherein the system is adapted to a subcutaneous biosensor.
